(19) United States
(12) Patent Application Publication
(10) Pub. No.: US 2007/0179085 A1
(43) Pub. Date: Aug. 2, 2007

(54) MODULATION OF RHAMM-CAVEOLIN/LIPID RAFT INTERACTIONS TO AFFECT DEVELOPMENT, RESPONSES TO TISSUE INJURY, ANGIOGENESIS, TUMORIGENESIS, METASTASIS AND GROWTH FACTOR/CYTOKINE RESPONSES

(76) Inventor: Rashmin C. Savani, Dallas, TX (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/458,990

(22) Filed: Jul. 20, 2006

Related U.S. Application Data
(60) Provisional application No. 60/700,906, filed on Jul. 20, 2005.

(51) Int. Cl. G01N 33/567; A61K 38/16

(52) U.S. Cl. 514/8; 435/7.2

(57) ABSTRACT

Compositions and methods for identifying agents which modulate RHAMM-mediated biological activities are provided. Also disclosed are methods for inhibiting exacerbation of certain injuries via regulation of RHAMM-mediated cellular signaling.

This application claims priority under 35 U.S.C. §119(e) to U.S. Provisional Application 60/700,906 filed Jul. 20, 2005.

Pursuant to 35 U.S.C. §202(c) it is acknowledged that the U.S. Government has certain rights in the invention described, which was made in part with funds from the National Institutes of Health, Grant Numbers HL42672 and HL073896.

### FIELD OF THE INVENTION

This invention relates to the fields of cellular and lipid metabolism. More specifically, the invention provided compositions and methods which modulate cell migration, and transformation and oncogenesis.

### BACKGROUND OF THE INVENTION

Several publications and patent documents are cited throughout the specification in order to describe the state of the art to which this invention pertains. Each of these citations is incorporated herein by reference as though set forth in full.

Acute lung injury is characterized by an initial inflammatory response that precedes pulmonary fibrosis (1-3). Pulmonary fibrotic disorders such as Acute Respiratory Distress Syndrome (ARDS) in adults and Bronchopulmonary Dysplasia in infants are characterized by a chronic inflammatory state consisting largely of macrophages (4, 5). The prevailing model of acute lung injury asserts that injury to the epithelium results in the release of numerous inflammatory mediators promoting the influx initially of neutrophils and subsequently macrophages into sites of injury with further increases in cytokine production and modulation of the extracellular matrix, including fibronectin, elastin, hyaluronan and collagen (6-10).

Hyaluronan (hyaluronic acid, HA) is a non-sulfated glycosaminoglycan that consists of a polymer of repeating disaccharide units of N-acetyl glucosamine and glucuronic acid (11). Increased HA concentrations are found coincident with periods of rapid cell proliferation and migration such as developing, regenerating, and remodeling tissues, as well as in tumorigenesis (reviewed in 12). Specifically, increased recovery of HA has been shown in bronchoalveolar lavage (BAL) from fibrotic lung diseases such as sarcoidosis (12), occupational lung disorders (D), and ARDS (14). Interestingly, HA-binding peptides administered to rodents have been shown to inhibit macrophage cell motility and reduce inflammation and fibrosis during skin wound repair (15), in the carageenen model of cutaneous inflammation (16), and after acute lung injury (17). These data suggest that HA is an integral part of the cause rather than a consequence of the inflammatory response to tissue injury.

HA modifies cell behavior through interactions with at least two molecularly distinct cell-associated receptors. CD44, a widely distributed cell surface glycoprotein (18, 19), exists in several molecular forms derived by alternate splicing of a single mRNA and exhibits different HA binding characteristics depending upon the cell type and conditions involved (20). It has been implicated in lymphocyte homing to tissues (21), tumorigenesis (22), and is clearly expressed in tissues undergoing repair after wounding (23). In acute lung injury, CD44 appears to mediate fibroblast migration (24) and HA-induced gene expression in mouse alveolar macrophages (25). CD44-deficient mice challenged with noninfectious lung injury exhibit sustained infiltration of inflammatory cells within the alveolar interstitium, increased mortality and LMW HA accumulation at 14 days, as well as impaired clearance of neutrophils in association with decreased TGFβ3 activation (26). This phenotype was significantly reversed with bone marrow reconstitution from CD44^{+/+} mice, suggesting an important role for CD44 in HA clearance after lung injury and resolution of the inflammatory response following tissue injury (26).

Since recruitment of inflammatory cells to the lung after injury is not affected in the absence of CD44, and blockade of HA inhibits macrophage accumulation, an HA receptor other than CD44 must mediate inflammatory cell accumulation. RHAMM (Receptor for HA-Mediated Motility, CD 168) is a HA-receptor expressed at the cell surface that regulates cell locomotion and proliferation (15, 27-31). In several injury models, RHAMM and HA are over-expressed in macrophages (15, 31), fibroblasts (32), epithelial (15), and smooth muscle cells (29) responding to lung injury. We therefore focused on the expression and role of RHAMM in inflammation using the function-blocking anti-RHAMM antibody, R36. We hypothesized that RHAMM plays an important role in the recruitment of inflammatory cells to the lung after bleomycin injury.

Vascular diseases such as atherosclerosis, restenosis and chronic allograft rejection cause significant morbidity and mortality in developed nations with an increased incidence of stroke, myocardial infarction, late transplant failure and peripheral gangrene (40). The pathogenesis of atherosclerotic and restenotic lesions is thought to be initiated by endothelial injury, followed by adherence and degranulation of platelets and release of growth factors and cytokines at the site of injury. An early inflammatory response adds to the accumulation of growth factors that are critical to neointimal formation. Under influence of these growth factors, medial smooth muscle cells (SMC) proliferate and migrate across the internal elastic lamina to create a neointima. Here, these cells undergo further proliferation and produce extracellular matrix that results in narrowing of the vessel (reviewed in ref. 39).

Platelet-Derived Growth Factor (PDGF), a highly hydrophilic cationic glycoprotein in the same superfamily of growth factors as Nerve Growth Factor and Transforming Growth Factor-B, exists as four monomeric proteins, A, B, C and D (2, 18, 30). These dimerize to form at least five different secreted forms of PDGF: PDGF-AA, PDGF-AB. PDGF-BB, PDGF-CC and PDGF-DD. Of these, PDGF-BB is known to play an important role in vascular SMC migration and proliferation (18). It is produced by platelets, activated macrophages, endothelial cells, and is released by injured vascular SMC (3, 18). PDGF-BB is a powerful chemoattractant and also a potent mitogen for vascular SMC in vitro (18). Importance of this growth factor to neointimal formation is highlighted by data showing that treatment of animals with PDGF-BB after balloon catheter injury to the carotid artery augments neointimal thickness (23). Conversely, treatment of injured animals with anti-PDGF-BB antibody inhibits neointimal formation in the same model (15).

Caveolae are 50-100 nm flask-shaped invaginations found in cholesterol and sphingolipid rich microdomains of the plasma membrane of most terminally differentiated cells. The formation of caveolae is critically dependent upon caveolins, a family of three genes, caveolin-1,-2,-3. Caveolin-1 and caveolin-2 are widely distributed, often as hetero-oligomers, whereas caveolin-3 is specific to muscle cells. Caveolins regulate diverse signaling pathways, acting as negative regulators via a specific scaffolding domain. Further, proteins that interact with caveolin do so via a consensus caveolin binding motif, ΦXΦXXXΦXXΦ where Φ is any aromatic amino acid. Peptides mimicking both the scaffolding domain and the caveolin binding motif have been used to identify numerous caveolin-associated molecules including arc-like kinases, MAP kinases, and the PDGFβ-receptor. Although some controversy exists concerning the relative functionality of caveolae and other lipid microdomains, caveolae are thought to act as signal transduction hubs by providing compartmentalization of intracellular signaling molecules.

The glycosaminoglycan hyaluronan (HA) has been localized to caveolae in SMC. HA, a ubiquitous glycosaminoglycan polymer of alternating saccharide units of D-glucuronic acid and N-acetylglucosamine, has been implicated in embryonic development, tumor growth, and wound healing, with specific activities that regulate cell migration and proliferation (25, 26). Preliminary studies, both from our laboratory and others, have shown that HA regulates migration (43) and proliferation (22) of SMC in vitro. Further, HA is present in atherosclerotic plaques, and accumulates in association with proliferating SMC after balloon catheter injury to the rat carotid artery (38).

The biologic responses to HA appear to be mediated by specific cell-associated receptors, of which two have thus far been characterized well, namely CD44 and Receptor for HA-Mediated Motility (RHAMM) (44). The expression and role of HA and these receptors in vascular injury responses has recently been reviewed (5). The expression of CD44 is increased in SMC after vascular injury in the rat, and anti-CD44 antibody partially blocks SMC proliferation in vitro (22). CD44 plays a critical role in athersclerosis since it promotes macrophage recruitment to atherosclerotic lesions and is required for dedifferentiation of SMC to the synthetic state characteristic of neointimal formation. In CD44/ Apo E double knockout mice, lesions were reduced by 50-70% (Pure). RHAMM, an N-glycosylated protein expressed both at the cell surface and intra-cellularly, mediates SMC migration in response to single scratch wounding of mono layers in vitro (41, 43). However, RHAMM lacks an obvious signal peptide, and it does not appear to possess a transmembrane domain. Accordingly, the molecular basis for its localization remains undefined and a matter of controversy. Interestingly, previous data suggests an association of RHAMM and erk after PDGF-BB stimulation, and it has been suggested that RHAMM is a glycosyl-phosphotidyl inositol (GPI)-linked protein.

### SUMMARY OF THE INVENTION

The purpose and object of the invention are to modulate the responses of cells and tissues in a favorable manner with respect to fundamental biological processes that affect development, responses to tissue/cellular injury, angiogenesis, and tumorigenesis and metastasis. Specifically, we have discovered that the interaction of RHAMM, GPI anchored proteins and Caveolins (1, 2 and 3)/Lipid rafts are essential requirements for growth factor/cytokine signaling. Promotion of such interactions, for example by using agonist ligands (eg. Hyaluronan, HA) will augment cell signaling and improve responses (eg. in wound healing). Inhibition of these interactions will block abnormal wound healing (eg. Fibrosis, keloids), as well as angiogenesis and progression of tumors. We demonstrate that anti-RHAMM antibody inhibits RHAMM:Caveolin/Lipid raft interaction at the cell surface. We have also discovered that RHAMM gets to the cell surface by being chaperoned by GPI-anchored proteins (eg. UPAR). Intracellular interference of the interaction of RHAMM with these GPI-anchored proteins and/or caveolins prevents RHAMM from getting to the cell surface to allow interaction with caveolae/lipid rafts. This represents a novel mechanism of RHAMM inhibition that is unlike that mediated by antibodies or extracellular acting peptides. In addition, the use of antibodies carries added complications of activation of inflammatory processes that likely limit the value of this therapy. The use of extracellular acting peptides carries the risks of interfering with multiple processes, including but not limited to the induction of immune deficiency in the face of infection/sepsis. The use of the RHAMM caveolin-binding peptide can be more directed to specific sites, and because it acts intra-cellularly, will not carry the risk of such side effects.

### BRIEF DESCRIPTION OF THE DRAWINGS

FIG. 1: Localization of RHAMM after intratracheal treatments RHAMM distribution was determined using diaminobenzadine-based immunohistochemistry using R36. (a) Uninjured control lung section stained with R36 pre-incubated with recombinant RHAMM protein shows no staining, confirming antibody specificity. (b) Normal RHAMM expression (brown staining) is restricted to the apex of airway epithelial cells, resident alveolar macrophages, and in bronchiolar smooth muscle. (c) At 4 days after injury, accumulating macrophages stain strongly for RHAMM. Macrophages were identified using the cell specific marker, ED1 (data not shown, see ref. (3 5)). Photomicrographs shown are representative of at least 5 animals examined at each time and for each condition.

FIG. 2: Immunoblot analysis of RHAMM after intratracheal treatments a). Expression of RHAMM was assessed in macrophages obtained by bronchoalveolar lavage. Equal protein was loaded from samples obtained from uninjured animals and compared to those from rats given either IT saline or bleomycin. Constitutive expression of 70 kDa and 80 kDa forms of RHAMM were noted. Densitometric analysis was performed as described in Methods. The expression of both isoforms increased after IT bleomycin and both forms were maximally expressed at 7 days. Animals given IT saline did not increase expression of either form of RHAMM. Blot shown is representative of three separate experiments showing similar results. b). In order to determine surface expression of RHAMM, macrophages obtained by lavage were cultured for two hours, then surface labeled with biotin. Cell lysates were then obtained and immunoprecipitated with streptavidin-sepharose. Subsequent immunoblotting for RHAMM showed that bleomycin injury was associated with a 2-fold increase in surface expression of the 70-kDa form and that the 80-kDa form was not expressed on the cell surface. Blot shown is representative of three separate experiments.

FIG. 3: In situ hybridization for RHAMM at 4 days Single strand digoxigenin-labeled DNA probes were used for non-radioactive in situ hybridization. Panel A and B are both sections from uninjured control animals. (a) Lack of hybridization using sense DNA on tissue sections from an uninjured control animal confirmed probe specificity. (b) Anti-sense RHAMM probe hybridized to resident alveolar macrophages (arrows). (c) 4 days after injury, anti-sense probe hybridized intensely to accumulating macrophages (arrowheads) suggesting that these cells are the site of RHAMM synthesis.

FIG. 4: Effect of anti-RHAMM antibody on macrophage motility 4 days after treatments (a). Timelapse cinemicrography was used to determine the effect of R36 on macrophage motility. Cells isolated from lavage of injured animals were plated and adherent macrophages were followed for 2 hours after the addition of 5 μg/ml of R36. Non-immune IgG was used as a control. Saline treatment alone caused some increase in macrophage motility. Bleomycin treatment resulted in a 5-fold increase in motility of macrophages. In both cases, motility was inhibited to baseline with R36 treatment, but not by non-immune IgG. These data suggest that RHAMM fully accounts for increased macrophage motility after intratracheal treatment. (b). HA oligosaccharide-stimulated chemotaxis of RAW 264.7 cells was assessed using a modified Boyden chamber assay. HA-6 stimulated a 2.5-fold increase in macrophage chemotaxis that was completely blocked in the presence of R36, but not with normal rabbit IgG (P<0.05, n=6/condition, representative experiment of three repeats)

FIG. 5: In vivo effect of anti-RHAMM antibody on lavage HA content and macrophage accumulation The effect of R36 on macrophage accumulation was determined by staining tissue sections with the rat macrophage specific marker ED 1. Three blinded observers independently counted positive cells per high power field (hpf). Saline treatment resulted in a small trend toward increased macrophage number as compared to controls (P=0.07). Bleomycin treatment resulted in a 3-fold increase in macrophage number (P<0.05 vs. saline). While normal IgG demonstrated non-specific effects, daily R36 treatment resulted in a further significant reduction in macrophage number after injury (p<0.05 vs. bleomycin+IgG).

FIG. 6: Effect of R36 on Lung Architecture at 7 days Fluorescent images of paraffin tissue sections illustrate that (A) saline treated animals had adequate alveoli and normal architecture. (B) Bleomycin treatment resulted in an influx of inflammatory cells with thickening of alveolar septae and early fibrosis. (C) Non-immune IgG also resulted in accumulation of inflammatory cells with evidence of fibrosis. (D) However, treatment with R36 resulted in decreased inflammatory cell accumulation and a return to more normal architecture. Data shown are representative of at least three animals examined for each condition.

FIG. 7: Stimulation of SMC migration by HA and PDGF-BB requires RHAMM A) A graph showing addition of HA to wounded cultures further stimulates migration of SMCs. B) A graph showing that the HA-stimulated migration is dose dependent. C) A graph showing the effect of PDGF-BB treatment and role of RHAMM in SMC migration after wounding. D) A graph showing that the effect of PDGF-BB on SMC proliferation is dose dependent. E) A graph showing the effect of HA on SMC proliferation and modulation by PDGF-BB.

FIG. 8: RHAMM associates with lipid domains and caveolins upon PDGF-BB stimulation A) Immunoblot analysis and non-detergent gradient fractionation on a time course study of quiescent vs. PDGF-BB treated SMC cultures. B) A western blot showing the distribution of RHAMM and caveolin in the presence and absence of PDGF-BB.

FIG. 9: Localization of RHAMM and caveolin-1 in the presence and absence of PDGF-BB treatment A) Quiescent, untreated SMC expressed caveolin-1 (red) on their surface, and RHAMM (green) expression was primarily intracellular.). With 6 hour PDGF-BB treatment, both RHAMM and caveolin-1 expression was increased intracellularly as well as on the cell surface, and extensive co-localization (yellow) was observed at the cell membrane. B) This co-localization did not occur at focal adhesions since no overlay was observed with vinculin (purple) even though focal adhesions were increased with PDGF-BB treatment. C) Immunoblot analysis for RHAMM showed that the 70 kda form of RHAMM co-precipitated with caveolin-1 antibody and that this association increased with PDGF-BB treatment.

FIG. 10: RHAMM is a glycosyl-phosphatidyl inositol anchored protein A) Immunoblot analysis of untreated SMC lysates showed that 70 kda RHAMM separated to the pellet phase, indicating that it is GPI-anchored. B) Immunoblot analysis showed that 70 kDa RHAMM was indeed cleaved and released into the media with PI-PLC treatment. C) A blot showing that co-treatment of cells with PDGF-BB and mannosamine for six hours completely inhibited the presence of RHAMM in the pellet phase after Triton X-114 triple fractionation.

FIG. 11: Movement of RHAMM within lipid raft subdomains A) Without MBCD, RHAMM was not released into the supernatant in detectable amounts, irrespective of PDGF-BB treatment compare lanes 2 vs 4). With MBCD treatment in the absence of PDGF-BB treatment (lane 3) 70 kDa RHAMM was released into supernatant. B) Immunoblot showing the release of RHAMM with MBCD with and without PDGF-BB treatment. R36 treatment of cultures was associated with continued MBCD-mediated release of RHAMM even in the face of PDGF-BB treatment, suggesting that R36 prevented the movement of RHAMM into the central sphingolipid core.

FIG. 12: Tthe effect of antibody and peptide treatment on erk phosphorylation. PDGF-BB stimulated SMC cultures A) Whole cell lysate was collected and immunoblotted for total and phosphorylated-erk. PDGF-BB treatment resulted in increased erk phosphorylation both in untreated and IgG-treated cultures, but not in R36-treated cultures

### DETAILED DESCRIPTION OF THE INVENTION

Lung injury is associated with increased concentrations of hyaluronan (hyaluronic acid, HA). HA modifies cell behavior through interaction with cell-associated receptors such as RHAMM (Receptor for HA-Mediated Motility). Using a function blocking anti-RHAMM antibody (R36), we investigated the expression and role of RHAMM in the inflammatory response to intratracheal bleomycin in rats. Immunostaining showed increased expression of RHAMM in macrophages four to seven days after injury. Surface biotin labeling of cells isolated by lavage confirmed increased surface expression of a 70 kDa RHAMM after lung injury, and in situ hybridization demonstrated increased RHAMM mRNA in macrophages responding to injury. Timelapse cinemicrography demonstrated a 5-fold increase in motility of alveolar macrophages from bleomycin treated animals that was completely blocked by R36 in vitro. Further, HA-stimulated macrophage chemotaxis was also inhibited by R36. Daily administration of R36 to injured animals resulted in a 40% decrease in macrophage accumulation seven days after injury. Further, H&E staining of tissue sections showed that bleomycin-mediated changes in lung architecture were improved with R36 treatment. Taken together with previous results showing the inhibitory effects of HA-binding peptide on inflammation and fibrosis, we conclude that the interaction of RHAMM with HA is a critical component of the recruitment of inflammatory cells to the lung after injury.

We also investigated whether RHAMM is a GPI anchored protein and its potential role in regulating injury and PDGF-BB mediated SMC migration. We show here that RHAMM is a critical requirement for PDGF-BB stimulated migration but not proliferation, and that it localizes to the cell surface via GPI modification. Most importantly, we show that RHAMM localizes to specific cholesterol-rich lipid domains in the plasma membrane of SMC, and its movement to the central sphingolipid core and interaction with caveolins via a specific caveolin-binding motif in RHAMM is a key requirement for propagating PDGF-BB signals for SMC migration and MAPK activation. We believe these findings are important to the plethora of signaling events regulated by caveolins within lipid domains and potentially explain the involvement of RHAMM in cell migration, transformation and oncogenesis.

Thus, in accordance with the present invention, methods of modulating RHAMM:Caveolin:Lipid Raft interactions are provided. Such modulators may include small molecules, peptides and antibody fragments.

The phrase “tissue injury” as used herein includes, without limitation, cancer, brain injury, asphyxia, ischemia-reperfusion injury, sepsis, myocardial infarction, and renal injuries or disease.

The phrase “inflammatory cells” includes, for example, leucocytes, such as macrophages, granulocytes, lymphocytes, B cells, and T cells.

The Examples set forth below are provided to illustrate certain embodiments of the invention. They are not intended to limit the invention in any way. The following materials and methods are provided to facilitate practice of Example I.

### EXAMPLE I

### Expression and Role of the Hyaluronan Receptor RHAMM in Inflammation after Bleomycin Injury

HA appears to exert its biological effects through binding interactions with specific cell-associated receptors. A number of HA-binding proteins have been identified, and two molecularly distinct cell-surface receptors for HA have been characterized, namely CD44 and RHAMM (for receptor for hyaluronan-mediated motility). The data presented herein demonstrate that the interaction of RHAMM with HA appears to directly activate intracellular signals required to stimulate processes relevant to inflammation and wound healing. Specifically, preliminary in vitro studies have suggested potential roles for these two molecules in aspects of HA-dependent RHAMM signal transduction.

Reagents and Macrophage Cell Line:

A rabbit polyclonal anti-RHAMM antibody, R36, was raised against amino acids 585-605 encoded in the full length RHAMM cDNA (33, 34) and purified to IgG fraction. Its specificity for RHAMM has been demonstrated previously (28, 29). A six-mer hyaluronan oligosaccharide (HA-6) was the kind gift of Dr. Akira Asari (Seikagaku Corporation, Tokyo, Japan). This oligosaccharide is produced by enzymatic digestion of high molecular weight HA extracted from rooster comb and purified using HPLC. HA-6 was demonstrated to be free of endotoxin, protein and DNA, and the molecular size and purity were confirmed by Fluorescence-Assisted Carbohydrate Electrophoresis (FACE) analysis and gas chromatography/mass spectroscopy (data not shown).

RAW 264.7 murine macrophages were obtained from ATCC (Cat# TIB-71, Rockville, Md.). Cells were maintained in DMEM supplemented with 10% heat-inactivated FBS, 2 mM L glutamine, 100 μg/ml streptomycin, and 100 U/ml penicillin at 37° C. under 5% CO₂.

Animals:

The Institutional Animal Care and Utilization Committees at both The Childrenls Hospital of Philadelphia and The University of Pennsylvania School of Medicine approved all animal care procedures. Six-week old (200-250 grams) male Sprague-Dawley rats (Charles River Breeding Laboratories, N. Wilmington, Mass.) were housed in the Animal Care Facility of The Children's Hospital of Philadelphia under standard conditions with free access to food and water. Animals were anesthetized with ketamine:xylazine:atropine (16:8:0.01 mg/kg), and the trachea was visualized through a vertical incision in the neck. Using an insulin syringe, 250 μl of either LPS-free saline or 8 units/kg bleomycin sulfate (Bristol Myers Squibb, Princeton, N.J.) in 250 μl of saline was injected into the trachea. The incision was closed with surgical clips, and animals were immediately injected intraperitoneally with either 0.5 mg of anti-RHAMM antibody (R36) or non-immune IgG. Antibody treatment was continued with daily injections of 0.5 mg of either R36 or IgG for either three or six days. At least three animals were examined for each condition, including one set that remained unmanipulated throughout.

Animals were sacrificed on days 4 or 7 after injury. Bronchoalveolar lavage (BAL) was obtained (36 ml/kg, total lung capacity) and immediately centrifuged at 500 g for 10 minutes to remove all cells and cellular debris. Approximately 80-90% of lavage was routinely recovered. The cellular pellet and the supernatant were separated and frozen at −70° C. until further analysis. The pulmonary artery was perfused with phosphate-buffered saline to remove all blood from the lungs. The left lung was either inflated to 25 cm H₂0 with 1% paraformaldehyde and placed in 10% neutral formalin for full fixation prior to processing for paraffin sectioning, or inflated with OCT/PBS solution to 25 cm H₂0 and immediately blocked in OCT and stored at −70° C. for frozen sectioning. The three lobes of the right lung were immediately frozen in liquid nitrogen and stored at −70° C.

Immunostaining:

RHAMM expression after bleomycin injury was determined using the Rabbit IgG Vectastain ABC kit (Vector Laboratories, Inc., Burlingame, Calif.) as previously described (17). Briefly, after removal of paraffin and stepwise hydration, slides were blocked with goat serum per manufacturer instructions. Primary antibody (R36, 25 μg/ml) was applied overnight at 4° C. in a humid slide box. Following washes in 0.01 M TBS and 0.01M TBS/0.1% Tween, endogenous peroxidase activity was blocked with 0.6% hydrogen peroxide/methanol for 30 minutes at room temperature. Secondary antibody, biotinylated goat anti-rabbit IgG, was applied for one hour at room temperature, and specific staining was obtained with avidin-biotin complex (ABC) and diaminobenzadine (DAB). Staining was enhanced with 0.5% CuSO₄ in 0.9% NaCl and slides were counterstained with 0.25% methyl green in ddH₂O. Slides were dipped quickly in n-Butanol, then xylene and finally mounted with Permount (Fisher Scientific, Fair Lawn, N.J.). Frozen sections were processed similarly except that they did not require removal of paraffin and rehydration, and were studied using immunofluorescence. Experiments to determine the changes in expression of RHAMM over time after intratracheal treatements were repeated three times. Densitometric analysis was used to quantify the 70 kDa and 80 kDa bands as described previously (35).

Macrophage accumulation was quantified by immunofluorescent staining with the rat macrophage specific marker ED1 (Serotec, Raleigh, N.C.). Endogenous fluorescence was blocked with separate exposure to sodium borohydrate (1 g/10 ml PBS, 3 minutes×2) and 1 M glycine in PBS for 30 minutes. Non-specific sites were blocked with PBS/100% goat serum/0.02% azide for 30 minutes at room temperature. Sections were incubated with fluorescein isothiocyanate (FITC)-conjugated ED1 overnight at 4° C. Slides were dried and mounted with Fluoromount-G (Southern Biotechnology Associates, Birmingham, Ala.). Three blinded, independent observers counted ED 1 positive cells per high power field to quantify macrophage accumulation in at least three lung sections from each animal. Each observer selected 3 random fields from different portions of the lung for counting and used sections obtained from different areas of lung tissue to ensure adequate sampling. In addition, all sections were observed under low power magnification to ensure that all areas of the lung section had been included in the determinations. Good concordance of results between the observers provided reassurance of the accuracy of the counting.

Immunoblot Analysis:

Western blots were performed for RHAMM in lysates of macrophages obtained by bronchoalveolar lavage. Isolated cells were incubated for 10 min with lysis buffer (25 mM Tris-HCl, 0.15 M NaCl, 1.0 mM EDTA, 1% sodium deoxycholate, 1% Triton X-100, 0.1% SDS, and Sigma protease inhibitor at 1:20 dilution). Cells were then scraped, transferred to microcentrifuge tubes, centrifuged at 14000 rpm for 10 min, and the resulting supernatant collected and stored at 80° C. The protein content of each sample was determined using the Bradford assay (36) and 10 μg of each sample was loaded and subjected to electrophoresis at 150 mV in Novex NuPAGE 12% Bis-Tris gels (Invitrogen, Carlsbad, Calif.) in MES-SDS buffer (from 40× stock from Invitrogen), transferred to nitrocellulose membranes, blocked with 5% non-fat dry milk (NFDM) reconstituted in Tris-buffered saline with 0.1% Tween 20 (TTBS) for one hour at room temperature, and probed with R36 (5 μg/ml) diluted in 2% NFDM-TTBS overnight at 4° C. After probing with the secondary antibody conjugated to horseradish peroxidase, protein bands were detected by enhanced chemiluminescence (Amersham, Piscataway, N.J.). Semiquantitative densitometry was performed on the resulting films with MacBASE version 2.4 (FUJIFilm, Elmsford, N.Y.) as described previously (35).

Surface Biotin Labeling:

Lavage cells were isolated from bronchoalveolar lavage by centrifugation at 5000 g for 5 minutes. Pelleted cells were resuspended in DMEM with no FBS. Cells were plated equally and macrophages adhered after 10 min at 37° C./5%CO₂. Surface biotin labeling (Pierce) was performed 2 hours after plating per manufacturer's instructions. Whole cell lysate was collected and 25 μg of each sample was immunoprecipitated with streptavidin-agarose as per manufacturer's instructions. The precipitate was resuspended in NuPAGE LDS sample buffer with DTT (Invitrogen) and separated by gel electrophoresis. Proteins in the gel were transferred to nitrocellulose membranes and immunoblots were performed for RHAMM as described above.

Probe Generation for in situ Hybridization

A partial cDNA for rat RHAMM was amplified from a rat smooth muscle cell cDNA library (the kind gift of Dr. C. Giachelli, The University of Washington, Seattle) by PCR using primers (5′: GTT GGT TGG TTG GAA AAA TCT; 3′: GCA GCA GTT CGG GTT GCC TTC TTT CAA) specific for positions 13 to 1645 of the cDNA (GenBank Accession No. U87983). The amplification was carried out using a hot start Mg⁺⁺ Bead (Invitrogen, San Diego Calif.) with a final concentration of 200 μM dNTPs, 1.5 mM Mg, 0.2 mM primers, 50 mM KCl, 10 mM Tris HCl, pH 8.3 and 0.25 Taq polymerase (Gibco BRL). Amplification was for 30 cycles on a programmable thermal cycler PTC-100 with a denaturation temperature of 94° C. for 1 minute, annealing temperature of 60° C. for 1 minute, and extension temperature of 72° C. for 2 minutes, and a terminal extension at 72° C. for 5 minutes. An aliquot was run on a 1% low melting point agarose gel, and the single band obtained was purified using the Wizard Mini Prep system (Promega). To generate sense and antisense single stranded digoxigenin-labelled probes, 20 ng of the initial PCR product was re-amplified with a single primer using 50 μM of dATP, dCTP, dGTP, 37 μM dTTP and 12.5 μM digoxigenin-labelled dUTP using the same amplification parameters and 50 cycles. A single band was generated on agarose gel and was purified again by Wizard Mini Prep. The single stranded probe generated by amplification with the 5′ primer (5′ GAA ATA GAA GAT CTT AAA CTG GAG AAT TTG 3′) generated a sense strand, while the 3′ primer (5′ CAA ATT CTC CAG TTT AAG ATC TTC TAT TTC 3′) generated an antisense probe (both from position 1067 of the rat RHAMM cDNA). This protocol is a modification of the technique described by Finckh et al. (37).

In situ Hybridization

Immediately after harvest, small portions of lung tissue were fixed in 4% paraformaldehyde in phosphate-buffered saline with freshly added 0.1% DEPC. After two hours, the tissue was cryoprotected in 30% sucrose overnight and then blocked in OCT (Miles). Four μM sections were cut and placed on silanated ProbeOn Slides (Fisher). In situ hybridization was performed following conventional protocols with the exception of the use of the Microprobe apparatus (Fisher) and 0.1% Brij 35 detergent in all pre-hybridization and post-hybridization steps. High stringency post-hybridization washes were with 0.1× SSC at room temperature 2 minutes per wash for 7 washes. The overnight hybridization was performed at 42° C. under coverslips to reduce the volume of reagents. Detection was with NBT-BCIP performed for 4 hours. At least two animals were studied for each treatment group.

Timelapse Cinemicrography:

Freshly isolated cells from bronchoalveolar lavage of saline or bleomycin-treated animals were monitored for their motility using a Nikon TE-300 inverted microscope (Nikon Corp., Tokyo, Japan) to which a video camera (Hamamatsu CCD, Inc., Hamamatsu City, Japan) was attached. Cell locomotion of at least 20 cells per animal was determined using Metamorph (Universal Imaging Corp., Downingtown, Pa.). Cells were plated and maintained at 37° C. and followed for 2 hours with mean velocities calculated every 10 minutes. The effect of anti-RHAMM antibody was examined by adding 5 μg/ml of antibody per plate per treatment 15 minutes before measurement of motility. Non-immune IgG was used as a control. Each experiment was repeated at least three times.

Chemotaxis Assay:

Chemotaxis of macrophages was performed using a modified Boyden chemotaxis chamber containing a 96-well microchemotaxis plate (MBA-96, Nuero Probe, Cabin John, MD) as described previously (38) with minor modifications. Briefly, the bottom wells of the chamber contained 40 μl of HA-6 (2 mM) dissolved in defined medium (DM), without fetal calf serum (FCS). Positive and negative controls included 10% FCS or DM respectively. The upper wells were filled with 1×10⁶ cells/ml suspended in 100 μl DM in the presence or absence of R36 or normal rabbit IgG. A 5 μm pore polycarbonate membrane filter was placed between the bottom and top chamber. The chamber was incubated for 6 hours at 37° C. Non-migratory cells on the upper surface of the membrane were treated with 200 μl of 1 mM EDTA for 15-20 min and wiped off. Cells that had migrated into the membrane were stained with Diff-Quick™ and counted in 5 randomly selected high power fields in each well. Each chemoattractant solution was tested in 6 wells and each experiment was repeated at least three times. Data were expressed as the number of macrophages that migrated into the membrane for each condition and converted to a percentage of control (DM) and combined for three separate experiments.

Statistical Analysis:

Statistical comparisons between uninjured animals, and saline or bleomycin-treated groups were carried out using ANOVA with Bonferroni correction for individual comparisons. All P values less than 0.05 were considered significant.

### Results

RHAMM Expression Increases in Macrophages after Bleomycin Lung Injury

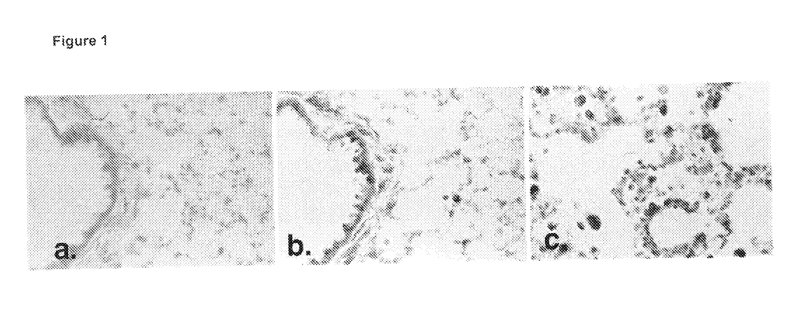

To determine the changes in RHAMM expression in the lungs of animals following intratracheal bleomycin, we first examined lung tissue sections by immunocytochemistry at various time points following injury (FIG. 1). Constitutive expression of RHAMM was observed in the bronchiolar epithelium, smooth musles, and resident alveolar macrophages (FIG. 1b). Four days after injury, staining for RHAMM was observed in macrophages accumulating both in the alveolar walls and in the alveolar spaces (FIG. 1c). In contrast, saline-instilled rat lungs were identical to uninjured, control lungs (data not shown). To ensure specificity of immunostaining, sections were incubated with antibody that had been pre-incubated with three-fold excess RHAMM fusion protein (FIG. 1a), or by the use of antibody that had been passed over a GST-RHAMM fusion protein column to remove RHAMM-specific antibody (data not shown). In both cases, staining was almost completely abrogated.

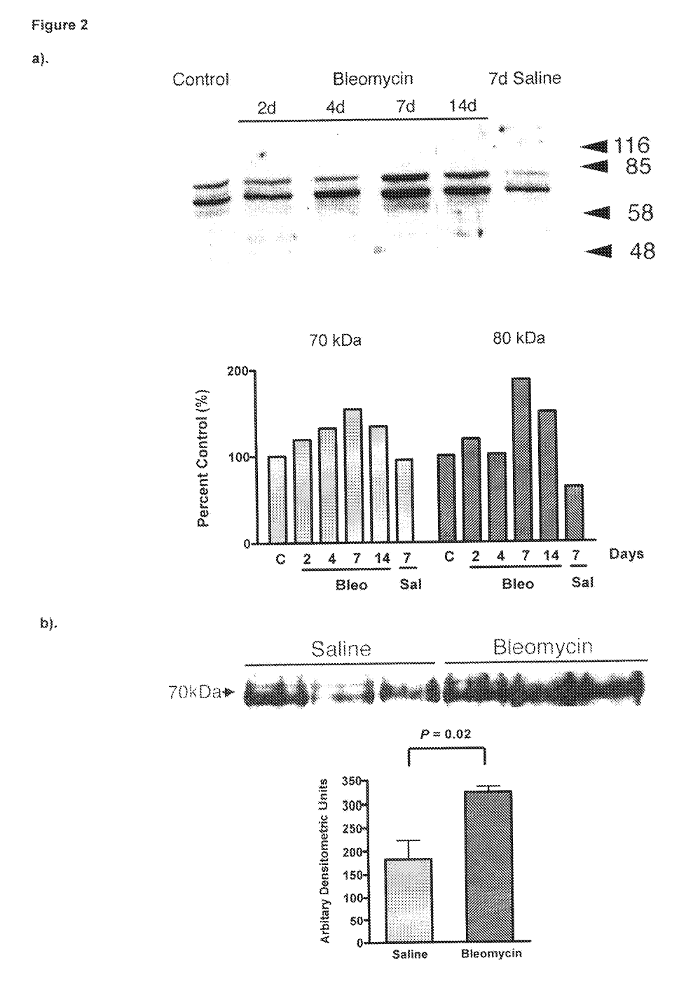

We next examined the expression of RHAMM in inflammatory cells obtained by lavage as a function of time after intratracheal treatments. Immunoblot analysis using R36 demonstrated 70 kDa and 80 kDa bands for RHAMM (FIG. 2a). Since equal protein was loaded for each lane, data represents the changes in RHAMM independent of the changes in numbers of cells accumulating after intratracheal treatments. Densitometric analysis of each band showed that bleomycin, but not saline treatment resulted in an increase in both isoforms to a maximum 7 days after bleomycin injury (FIG. 2a).

Since cell surface expression of RHAMM has been correlated with increased cell motility (29, 39), and we had previously shown increased surface expression of RHAMM by flow cytometry of lavage cells from bleomycin-injured animals (31), we also examined the expression of cell surface RHAMM by surface biotin labeling seven days after intratracheal treatments. Compared to saline-treated rats, a 2-fold increase in the 70 kDa band was observed in lavage macrophages at seven days after bleomycin indicating the upregulation of the 70 Kda RHAMM isoform at the cell surface following bleomycin injury (FIG. 2b, P=0.02, n=3).

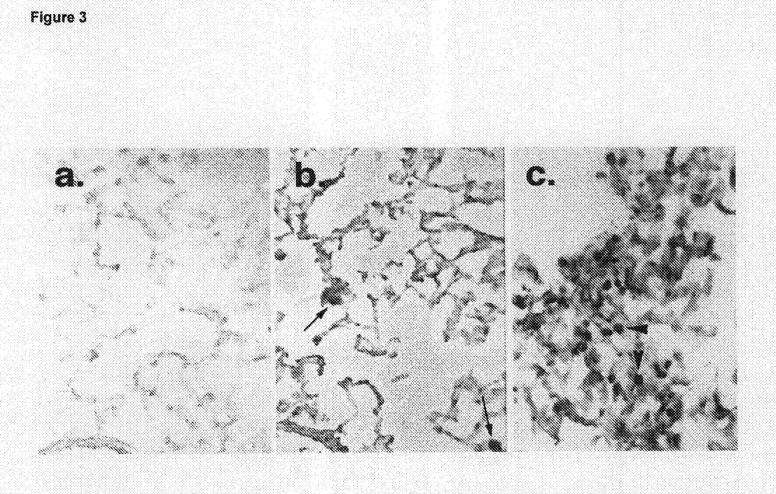

In order to determine the cells responsible for synthesizing RHAMM, in situ hybridization was employed. Single stranded sense and antisense DNA probes were used in a non-radioactive digoxigenin-labelled in situ hybridization. Consistent with the immunohistochemical data, four days after intratracheal saline administration RHAMM mRNA was localized to resident alveolar macrophages (FIG. 3b). This distribution did not differ from that of uninjured normal rat lungs (data not shown). Intense positive staining with the antisense probe was obtained in macrophages accumulating in areas of lung injury four days after bleomycin injury, confirming that these cells were the sites of RHAMM synthesis (FIG. 3c). No staining was observed with the sense probe thereby confirming the specificity of the antisense probe (FIG. 3a).

RHAMM is Necessary for Increased Macrophage Motility after Injury

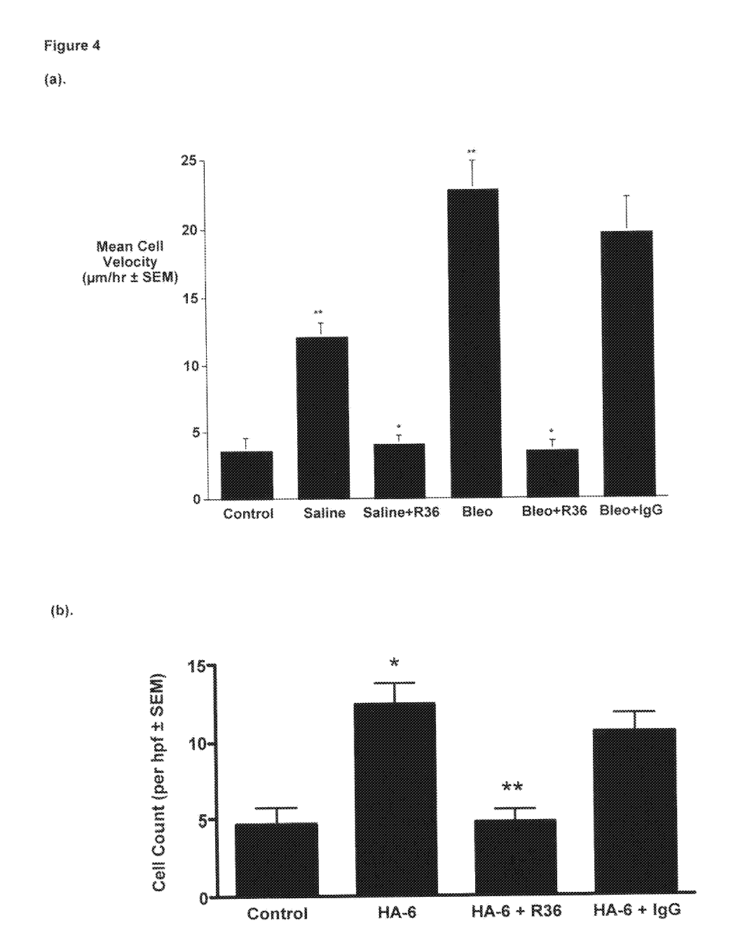

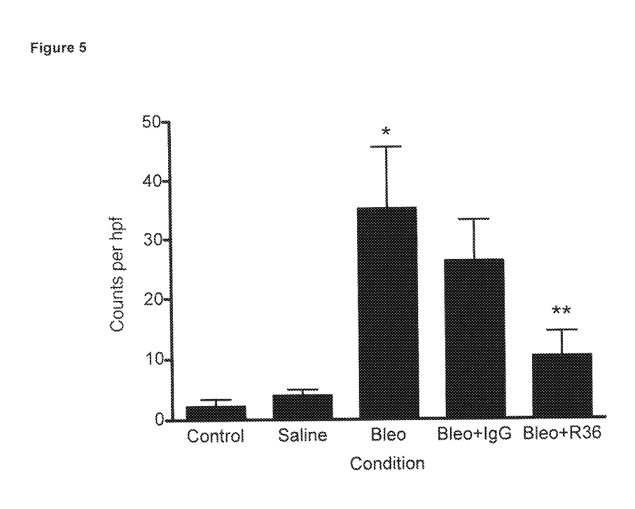

To determine whether the increased expression of RHAMM observed in macrophages four days after injury corresponded to changes in motility, we used time-lapse cinemicrography to measure the locomotion of macrophages in the first two hours after isolation by BAL from control and bleomycin-treated animals four days after intratracheal treatments (FIG. 4). Macrophages after bleomycin treatment showed significantly higher velocities than saline-treated controls (FIG. 4, **P<0.001, n=4). Macrophages isolated from saline-treated animals also showed a small increase in cell locomotion as compared to untreated controls (FIG. 4, *P<0.05, n=4). This may represent injury as a result of instillation of saline into the lungs. In both saline- and bleomycin-treated animals, treatment with R36 inhibited increased motility to baseline (FIG. 4, * P<0.05, n=4). Non-immune IgG, used as a control, had no effect on the motility of cells from bleomycin-treated animals. These data suggest that, as described for HA (17), RHAMM fully accounts for increased macrophage motility observed after bleomycin injury.

RHAMM is Necessary for HA-mediated Macrophage Chemotaxis

In order to determine whether RHAMM contributes to HA-stimulated chemotaxis, a modified Boyden chamber assay was performed using a six-mer HA oligosaccharide (HA-6) as the chemoattractant for RAW264.7 murine macrophages as described in Methods. HA-6 stimulated a 2.5-fold increase in macrophage chemotaxis over defined medium as a control (FIG. 4b, *P<0.01, n=6 per condition, experiment repeated three times). Addition of R36 to the top chamber with the cells inhibited HA-stimulated chemotaxis to baseline (FIG. 4b, **P<0.01 vs. HA-6 and HA-6+Rabbit IgG, n=6 per condition, repeated three times), whereas normal rabbit IgG had no effect. These data suggest that in addition to the random migration of macrophages, RHAMM:HA interactions mediate macrophage chemotaxis.

In vivo Effects of RHAMM Antibody

We next evaluated the effects of R36 on the inflammatory response to bleomycin. To quantify the effect of R36 on macrophage accumulation, tissue sections were immunostained with ED1 and positive cells per high power field (hpf) were counted (FIG. 5). Saline treatment resulted in a trend to higher macrophage number as compared to controls (saline 14.9±3.3 vs. control 10.9±0.21 cells per hpf, n=4, p=0.07). However, bleomycin treatment resulted in a 3-fold increase in macrophage number (28.4±4.7 cells per hpf, P<0.05, n=4). Daily R36 treatment resulted in a 40% reduction in macrophage number after injury as compared to non-immune IgG-treated animals (FIG. 5, 17.7±4.9 cells per hpf, P<0.05, n=-4).

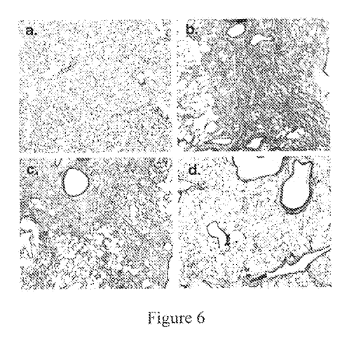

Finally, we determined the effect of antibody treatment on lung architecture at seven days. Saline treated animals showed normal lung architecture (FIG. 6a). Bleomycin treatment resulted in an influx of inflammatory cells with thickening of alveolar septae and early fibrosis (FIG. 6b). Non-immune IgG also resulted in accumulation of inflammatory cells with evidence of fibrosis (FIG. 6c). However, treatment with R36 resulted in decreased inflammatory cell accumulation and a return to more normal architecture (FIG. 6d).

### Discussion

In the present study, we demonstrate increased synthesis and expression of the pro-migratory molecule RHAMM in macrophages accumulating in areas of the lung injured by intratracheal instillation of bleomycin. Elevated expression of RHAMM occurred in association with increased macrophage motility observed after injury. In addition, using antibody inhibition, we show that cell surface RHAMM is not only necessary for this increased motility, but also contributes to lung macrophage accumulation after injury. Taken together with our previous studies demonstrating the ability of HA-binding peptide to decrease inflammation and fibrosis after bleomycin injury (17), the results of the current study suggest that RHAMM:HA interactions play a critical role in the recruitment of inflammatory cells after lung injury by bleomycin.

HA exerts direct effects on cells and on the extracellular matrix that may be relevant to its role in wound repair. Thus, the proinflammatory cytokines TNFα and IL-1 β have been shown to induce cell surface expression of HA (40) that in turn promotes adhesion of leukocytes to the endothelium (40, 41). Further, HA has been shown to stimulate the migration and proliferation of smooth muscle cells (29), fibroblasts (27), immune cells (15), and endothelial cells (42, 43). HA also activates monocytes into macrophages (44), and increases cytokine gene expression by macrophages (25) and fibroblasts (45). Increased accumulation of HA is associated with inflammation following acute injury to several organ systems including the lung (reviewed in Savani et al., (31) and (10)). However, recent studies have suggested that HA promotes inflammation. Thus, HA-binding peptide specifically blocks macrophage motility in vitro and, when systemically administered to rats during bleomycin injury, results in decreased alveolar macrophage motility and accumulation with subsequently reduced collagen content in the lung (17). In rats, subcutaneous injection of HA-binding peptide before mechanical ventilation blocks neutrophil influx in the lung responding to intratracheal administration of LMW HA (46). Importance of HA to inflammation and the likely universal nature of this response was confirmed by Mummert et al. (16). In a model of contact hypersensitivity, this group demonstrated that systemic, local, or topical administration of a different HA-binding peptide to reactive hapten-sensitized mice blocked skin-directed homing of inflammatory leukocytes. Together, these studies suggest that the interaction of HA with its receptors precedes and promotes the inflammatory response to injury.

Several other authors have also demonstrated increased cell surface RHAMM expression in leukocytes (47-49). Further, we have previously shown that inflammatory cell chemotaxis and random migration is dependent on RHAMM (15). Interestingly, RHAMM expression is unaltered in CD44 null mice that exhibit increased inflammation after collagen-induced arthritis and anti-RHAMM antibody blocks the inflammatory cell influx in the absence of CD44 (50). Thus, RHAMM:HA interactions may be responsible for leukocyte transmigration through the endothelium and for chemotaxis within injured areas of the lung. However, other reports have noted an absence of cell surface RHAMM after injury. Teder et al. (51, 52) proposed two mechanisms for the increased accumulation of HA after bleomycin injury in rats. First, they showed that fibroblasts exposed to BAL from injured animals increase their production of HA, a response that was largely abrogated by blocking antibodies to TGF-β1 (51). Further, alveolar macrophages obtained five days after bleomycin injury bound less [³H]-hyaluronan than those from saline treated controls, suggesting lower HA receptor expression in macrophages after injury (52). This lower expression of HA receptors was also thought to contribute to elevated HA since less HA would be internalized by these cells. Indeed, in their study, no RHAMM could be detected on the surface of these cells. Further, Weiss et al. (9) examined the HA-binding properties of activated monocytes. Immunostaining of skin samples from allergic contact dermatitis found little HA on vascular endothelial cells or activated lymphocytes and no RHAMM expression in monocytes infiltrating sites of cutaneous inflammation. In vitro activation of peripheral blood monocytes by tissue culture, plastic adhesion, and treatment with LPS and IFN-γ did increase HA binding, but did not upregulate RHAMM expression. Instead, FACS analysis showed that activated monocytes expressed increased cell surface CD44, coinciding with increased CD44 mRNA and protein (9).

The lack of increased HA receptor expression in macrophages after bleomycin injury as reported by Teder et al. (52) is contradictory to other reports and the data presented in the current study. For instance, the expression of the standard form of CD44 (CD44s) is increased in alveolar macrophages and in the interstitium of the lung in areas of thickened alveolar wall, while the expression of CD44v6, thought to be the epithelial form, is decreased in type II pneumocytes (53). Further, CD44, presumably acting thorough its ability to bind HA, has been implicated in novel rolling and adhesion pathways leading to leukocyte adhesion to activated endothelium (40, 41). Further, the recruitment of lymphocytes to the lung following antigenic challenge to sensitized mice could be blocked using an anti-CD44 antibody (54-57).

Thus, there are conflicting data with respect to increased expression of CD44 and RHAMM after lung injury. It is unclear as to why these differences in observations exist. However, it is relevant to this discrepancy that we have noticed that the increased motility observed in macrophages isolated from bleomycin-injured animals is rapidly lost after two hours following culture (data not shown). Since Teder and colleagues (52) examined macrophages after 24 hours in culture, the decreased surface expression of HA receptors observed could be the result of down-regulated cell surface expression of HA receptors.

Upregulation of growth factors such as TNF and TGFβ after bleomycin-induced lung injury coincides with increased HA production (58, 59). In lungs injured by bleomycin, the increased expression of HA receptors in macrophages coincides with the temporal increase in TGF-β1 expression observed in the same cells after similar injury (8, 53). This similarity in expression suggests in vivo regulation of HA receptors in macrophages by TGF-β1. In fibroblasts, the expression of RHAMM has been shown to be regulated by TGF-β1 by a mechanism that involves increased message stability (60), and the RHAMM:HA interaction is required for TGF-β1-stimulated increases in cell locomotion (61). TGF-β1 may thus be responsible for an autocrine system in which production of this growth factor results in upregulation of HA and its receptors in macrophages and a paracrine effect on fibroblasts to produce HA and increased collagen deposition, thereby influencing both inflammation and fibrosis after injury. Our data, however, showing a decrease in macrophage accumulation with HA-binding peptide treatment (17), and with anti-RHAMM antibody treatment (this study) suggests that RHAMM:HA interactions are, at least in part, responsible for the inflammatory response to acute lung injury.

In summary, the results of this study indicate that HA and RHAMM are critical components of the inflammatory and fibrotic processes resulting from acute lung injury and that these molecules represent novel targets to limit the adverse consequences of lung injury in humans.

### REFERENCES FOR EXAMPLE I

1. Selman, M., T. E. King Jr, and A. Pardo, 2001. Idiopathic Pulmonary Fibrosis: prevailing and evolving hypotheses about its pathogenesis and implications for therapy. Ann. Internal Med 134:136-151.
2. Downey, G. P., Q. Dong, J. Kruger, S. Dedhar, and V. Cherapanov. 1999. Regulation of neutrophil activation in acute lung injury. Chest 116(l):46S-54S.
3. Hasleton, P. S., and T. E. Roberts. 1999. Adult Respiratory Distress Syndrome: an update. Histopathology 34:285-294.
4. Ware, L. B., and M. A. Matthay. 2000. The acute respiratory distress syndrome. New Engl. J. Med 342(18):1334-1349.
5. Groneck, P., and C. P. Speer. 1997. Pulmonary inflammation in the pathogenesis of bronchopulmonary dysplasia. Pediatr. Pulmonol. 16((Suppl.)):29-30.
6. Raghow, R., S. Lurie, J. M. Seyer, and A. H. Kang. 1985. Profiles of steady state levels of messenger RNAs coding for Type I Procollagen, Elastin and Fibronectin in hamster lungs undergoing bleomycin-induced interstitial pulmonary fibrosis. J Clin. Invest. 76(November): 1733-1739.
7. Hernnas, J., O. Nettelbladt, L. Bjermer, B. Sarnstrand, A. Malmstrom, and R. Hällgren. 1992. Alveolar accumulation of fibronectin precedes fibrosis in bleomycin-induced pulmonary injury. Eur. Respir. J. 5(4):404-410.
8. Khalil, N., C. Whitman, L. Zuo, D. Danielpour, and A. Greenberg. 1993. Regulation of alveolar macrophage transforming growth factor-beta secretion by corticosteroids in bleomycin-induced pulmonary inflammation. J. Clin. Invest. 92(4):1812-1818.
9. Weiss, J. M., A. C. Renkl, T. Ahrens, J. Moll, B. H. Mal, R. W. Denfeld, E. Schopf, H. Ponta, P. Herrlich, and J. C. Simon. 1998. Activation-dependent modulation of hyaluronate-receptor expression and of hyaluronate avidity in human monocytes. J. Invest. Dermatol. 111(2):227-232.
10. Savani, R. C., and H. M. DeLisser. 2003. Hyaluronan and its receptors in lung health and disease. In H. G. Garg, P. J. Roughley and C. A. Hales, editors. Proteoglycans and Lung Disease, Lung Biology in Health and Disease. Marcel Dekker, New York. 73-106.
11. Laurent, T. C., and J. R. E. Fraser. 1992. Hyaluronan. FASEB J. 6:2397-2404.
12. Hällgren, R., A. Eklund, A. Engstrom-Laurent, and B. Schmekel. 1985. Hyaluronate in bronchoalveolar lavage fluid: a new marker in sarcoidosis reflecting pulmonary disease. Br. Med J. 290:1778-1781.
13. Bjermer, L., A. Engstrom-Laurent, R. Lundgren, L. Rosenhall, and R. Hallgren. 1987. Hyaluronate and type III procollagen peptide concentrations in bronchoalveolar lavage fluid as markers of disease activity in farmer's lung. Br. Med. J. 295:803-806.
14. Hällgren, R., T. Samuelsson, T. C. Laurent, and J. Modig. 1989. Accumulation of hyaluronan (hyaluronic acid) in the lung in Adult Respiratory Distress Syndrome. Am. Rev. Respir. Dis. 139:682-687.
15. Savani, R. C., N. Khalil, and E. A. Turley. 1995. Hyaluronan receptor antagonists alter skin inflammation and fibrosis following injury. Proc. W Pharmacol. Soc. 38:131-136.
16. Mummert, M. E., M. Mohamadzadeh, D. I. Mummert, N. Mizumoto, and A. Takashima. 2000. Development of a peptide inhibitor of hyaluronan-mediated leukocyte trafficking. J. Exp. Med 192(6):769-779.
17. Savani, R. C., G. Hou, P. Liu, C. Wang, E. Simons, P. C. Grimm, R. Stem, A. H. Greenberg, H. DeLisser, and N. Khalil. 2000. A Role for Hyaluronan (HA) in macrophage accumulation and collagen deposition after bleomycin-induced lung injury. Am. J. Respir. Cell Mol Biol. 23(4):475-484.
18. Entwistle, J., C. L. Hall, and E. A. Turley. 1996. HA Receptors: Regulators of cell signaling to the cytoskeleton. J. Cell. Biochem. 61:569-577.
19. Naor, D., R. V. Sionov, and D. Ish-Shalom. 1997. CD44: structure, function and association with the malignant process. Adv. Cancer Res. 71:241-319.
20. Day, A. J. 1999. The structure and regulation of hyaluronan-binding proteins. Biochem. Soc. Trans. 27(2):115-121.
21. Miyake, K., K. L. Medina, S.-I. Hayashi, S. Ono, T. Hamaoka, and P. W. Kincade. 1990. Monoclonal antibodies to Pgp-l/CD44 block lympho-hemopoiesis in long-term bone marrow cultures. J. Exp. Med 171 (February):477-488.
22. Salmi, M., K. Gron-Virta, P. Sointa, R. Grenman, H. Kalimo, and S. Jalkanen. 1993. Regulated expression of exon v6 containing isoforms of CD44 in man: downregulation during malignant transformation of tumors of squamocellular origin. J. Cell Biol. 122(2):431-442.
23. Penneys, N. S. 1993. CD44 expression in normal and inflamed skin. J. Cutan. Pathol. 20(3):250-253.
24. Svee, K., J. White, P. Vaillant, J. Jessurun, U. Roongta, M. Krumwide, D. Johnson, and C. Henke. 1996. Acute lung injury fibroblast migration and invasion of a fibrin matrix is mediated by CD44. J. Clin. Invest. 98:1713-1727.
25. McKee, C. M., M. B. Penno, M. Cowman, M. D. Burdick, R. M. Stricter, C. Bao, and P. W. Noble. 1996. Hyaluronan (HA) fragments induce chemokine gene expression in alveolar macrophages: The role of HA size and CD44. J. Clin. Invest. 98(10):2403-2413.
26. Teder, P., R. W. Vandivier, D. Jiang, J. Liang, L. Cohn, E. Pure, P. M. Henson, and P. W. Noble. 2002. Resolution of lung inflammation by CD44. Science 296(5565):155-158.
27. Hardwick, C., K. Hoare, R. Owens, H. P. Hohn, M. Hook, D. Moore, V. Cripps, L. Austen, D. M. Nance, and E. A. Turley. 1992. Molecular cloning of a novel Hyaluronan receptor that mediates tumor cell motility. J. Cell. Biol. 117(6):1343-1350.
28. Hall, C. L., B. Yang, X. Yang, S. Zhang, M. Turley, S. Samuel, L. A. Lange, C. Wang, G. D. Curpen, R. C. Savani, A. H. Greenberg, and E. A. Turley. 1995. Overexpression of the hyaluronan receptor RHAMM is transforming and is required for transformation by H-ras. Cell 82(1):19-28.
29. Savani, R. C., C. Wang, B. Yang, S. Zhang, M. G. Kinsella, T. N. Wight, R. Stem, D. M. Nance, and E. A. Turley. 1995. Migration of bovine aortic smooth muscle cells following wounding injury: The role of Hyaluronan and RHAMM. J. Clin. Invest. 95:1158-1168.
30. Zhang, S., M. C. Chang, D. Zylka, S. Turley, R. Harrison, and E. A. Turley. 1998. The hyaluronan receptor RHAMM regulates extracellular-regulated kinase. J Biol Chem 273(18):11342-8.
31. Savani, R. C., D. J. Bagli, R. E. Harrison, and E. A. Turley. 2000. The role of hyaluronan-receptor interactions in wound repair. In H. G. Garg and M. T. Longaker, editors. Scarless Wound Healing, Basic and Clinical Dermatology, 19 ed. Marcel Dekker, New York-Basel. 115-142.
32. Lovvorn, H. N., D. L. Cass, K. G. Sylvester, E. Y. Yang, T. M. Crombleholme, N. S. Adzick, and R. C. Savani. 1998. Hyaluronan receptor expression increases in fetal excisional skin wounds and correlates with fibroplasia. J. Pediatr. Surg. 33(7):1062-1070.
33. Entwistle, J., S. Zhang, B. Yang, C. Wong, L. Qun, C. L. Hall, A. Jingbo, M. Mowat, A. H. Greenberg, and E. A. Turley. 1995. Characterization of the murine gene encoding the hyaluronan receptor RHAMM. Gene 163:233-238.
34. Fieber, C., R. Plug, J. Sleeman, P. Dall, H. Ponta, and M. Hofmann. 1999. Characterization of the murine gene encoding the intracellular hyaluronan receptor IHABP (RHAMM). Gene 226(1):41-50.
35. Savani, R. C., R. I. Godinez, M. H. Godinez, E. Wentz, A. Zaman, Z. Cui, P. M. Pooler, S. H. Guttentag, M. F. Beers, L. W. Gonzales, and P. L. Ballard. 2001. Respiratory distress after intratracheal bleomycin: selective deficiency of surfactant proteins B and C. Am. J. Physiol. Lung Cell Mol. Physiol. 281 :L685-L696.
36. Bradford, M. M. 1976. A rapid and sensitive method for the quantitation of microgram quantities of protein utilizing the principle of protein-dye binding. Anal Biochem. 72:248-254.
37. Finckh, U., P. Lingenfelter, and D. Myerson. 1991. Producing single-stranded DNA probes with the Taq DNA polymerase: A high yield protocol. Biotechniques 10:35-39.
38. Shi, Y., B. S. Kornovski, R. C. Savani, and E. A. Turley. 1993. A rapid, multiwell colorometric assay for chemotaxis. J. Immunol. Methods 164:149-154.
39. Hall, C. L., C. Wang, L. A. Lange, and E. A. Turley. 1994. Hyaluronan and the hyaluronan receptor RHAMM promote focal adhesion turnover and transient tyrosine kinase turnover. J. Cell Biol. 126:575-588.
40. Mohamadzadeh, M., H. DeGrendele, H. Arizpe, P. Estess, and M. Siegelman. 1998. Proinflammatory stimuli regulate endothelial hyaluronan expression and CD44/HA-dependent primary adhesion. J. Clin. Invest. 101(1):97-108.
41. DeGrendele, H. C., P. Estess, L. J. Picker, and M. H. Siegelman. 1996. CD44 and its ligand hyaluronate mediate rolling under physiologic flow: a novel lymphocyte-endothelial cell primary adhesive pathway. J. Exp. Med. 183(3): 1119-1130.
42. Trochon, V., C. Mabilat, P. Bertrand, Y. Legrand, F. Smadja-Joffe, C. Soria, B. Delpech, and H. Lu. 1996. Evidence of involvement of CD44 in endothelial cell proliferation, migration and angiogenesis in vitro. Int. J. Cancer 66:664-668.
43. Savani, R. C., G. Cao, P. M. Pooler, A. Zaman, Z. Zhou, and H. M. DeLisser. 2001. Differential involvement of the hyaluronan (HA) receptors CD44 and Receptor for HA-Mediated Motility in endothelial cell function and angiogenesis. J. Biol. Chem. 276(39):36770-36778.
44. Noble, P. W., F. R. Lake, P. M. Henson, and D. W. Riches. 1993. Hyaluronate activation of CD44 induces insulin-like growth factor-1 expression by a tumor necrosis factor-alpha-dependent mechanism in murine macrophages. J. Clin. Invest. 91(6):2368-2377.
45. Kobayashi, H., and T. Terao. 1997. Hyaluronic acid-specific regulation of cytokines by human uterine fibroblasts. Am. J. Physiol. 273(4, Pt.1):C1151-C1159.
46. Syrkina, O. L., M. M. Mascarenhas, H. G. Garg, C. A. Hales, and D. A. Quinn. 2003. Neutrophil influx with ventilator-induced lung injury (VILI) is dependent on low molecular weight hyaluronic acid. Am. J. Respir. Crit. Care Med. 167(7):A563.
47. Doherty, D. E., N. Hirose, L. Zagarella, and R. M. Cherniack. 1992. Prolonged monocyte accumulation in the lung during bleomycin-induced pulmonary fibrosis: a non-invasive assesment of monocyte kinetics by scintigraphy. Lab. Invest. 66:231-242.
48. Bazil, V., and V. Horejsi. 1992. Shedding of the CD44 adhesion molecule from leukocytes induced by anti-CD44 monoclonal antibody stimulating the effect of a natural receptor ligand. J. Immunol. 149(3):747-753.
49. Pilarski, L. M., E. Pruski, J. Wizniak, D. Paine, K. Seeberger, M. J. Mant, C. B. Brown, and A. R. Belch. 1999. Potential role for hyaluronan and the hyaluronan receptor RHAMM in mobilization and trafficking of hematopoietic progenitor cells. Blood 93(9):2918-2927.
50. Nedvetzki, S., E. Gonen, N. Assayag, R. Reich, R. O. Williams, R. L. Thurmond, J. F. Huang, B. A. Neudecker, F. S. Wang, E. A. Turley, and D. Naor. 2004. RHAMM, a receptor for hyaluronan-mediated motility, compensates for CD44 in inflamed CD44-knockout mice: a different interpretation of redundancy. Proc Natl Acad Sci USA 101(52):18081-6.
51. Teder, P., O. Nettelbladt, and P. Heldin. 1995. Characterization of the mechanism involved in bleomycin-induced increased hyaluronan production in rat lung. Am. J. Respir. Cell Mol. Biot 12:181-189.
52. Teder, P., and P. Heldin. 1997. Mechanism of impaired local hyaluronan turnover in bleomycin-induced lung injury in rat. Am. J. Respir. Cell Mol. Biol. 17(3):376-385.
53. Kasper, M., A. Bierhaus, A. Whyte, R. M. Binns, D. Schuh, and M. Muller. 1996. Expression of CD44 isoforms during bleomycin- or radiation-induced pulmonary fibrosis in rats and mini-pigs. Histochem. Cell Biol. 105:221-230.
54. Curtis, J. L., S. Kim, P. J. Scott, and V. A. Buechner-Maxwell. 1995. Adhesion receptor phenotypes of the murine lung CD4+T cells during the pulmonary immune response to sheep erythrocytes. Am. J. Respir. Cell Mol. Biol. 12(5):520-530.
55. Wolber, F. M., J. L. Curtis, A. M. Milik, T. Fields, G. D. Seitzman, K. Kim, S. Kim, J. Sonstein, and L. M. Stoolman. 1997. Lymphocyte recruitment and kinetics of adhesion receptor expression during the pulmonary response to particulate antigen. Am. J. Pathol. 151(6):1715-1727.
56. Wolber, F. M., J. L. Curtis, P. Maly, J. Kelly, P. Smith, T. A. Yednock, J. B. Lowe, and L. M. Stoolman. 1998. Endothelial selecting and alpha-integrins regulate independent pathways of T lymphocyte recruitment in the pulmonary immune response. J. Immunol. 161(8):4396-4403.
57. Curtis, J. L., F. M. Wolber, J. Sonstein, R. A. Craig, T. Polak, R. N. Knibbs, J. Todt, G. D. Seitzman, and L. M. Stoolman. 2000. Lymphocyte-endothelia cell adhesive interactions in lung immunity: lessons from the murine reponse to particulate antigen. Immunopharmacology 48(3):223-229.
58. Heldin, P., T. C. Laurent, and C. H. Heldin. 1989. Effect of growth factors on hyaluronan synthesis in cultured human fibroblasts. Biochem. J. 258(3):919-922.
59. Heldin, P., T. Asplund, D. Ytterberg, S. Thelin, and T. C. Laurent. 1992. Characterization of the molecular mechanism involved in the activation of hyaluronan synthetase by platelet-derived growth factor in human mesothelial cells. Biochem. J. 283(1):165-70.
60. Amara, F. M., J. Entwistle, T. I. Kuschak, E. A. Turley, and J. A. Wright. 1996. Transforming growth factor-beta 1 stimulates multiple protein interactions at a unique cis-element in the 3′-untranslated region of the hyaluronan receptor RHAMM mRNA. J. Biol. Chem. 271(25):15279-15284.
61. Samuel, S. K., R. A. R. Hurta, M. A. Spearman, J. A. Wright, E. A. Turley, and A. H. Greenberg. 1993. TGF-β₁ stimulation of cell locomotion utilizes the hyaluronan receptor RHAMM and hyaluronan. J. Cell Biol. 123(3):749-758.

### EXAMPLE II

### Stimulation of SMC Migration by HA and PDGF-BB Requires RHAMM

The biologic responses to HA appear to be mediated by specific cell-associated receptors, of which two have thus far been characterized well, namely CD44 and Receptor for HA-Mediated Motility (RHAMM, CD168). The expression and role of HA and these receptors in vascular injury responses has recently been reviewed. The expression of CD44 is increased in SMC after vascular injury in the rat, and anti-CD44 antibody partially blocks SMC proliferation in vitro. CD44 plays a critical role in athersclerosis since it promotes macrophage recruitment to atherosclerotic lesions and is required for dedifferentiation of SMC to the synthetic state characteristic of neointimal formation. In CD44/Apo E double knockout mice, lesions were reduced by 50-70%. RHAMM, an N-glycosylated protein expressed both at the cell surface and intracellularly, mediates SMC migration in response to single scratch wounding of monolayers in vitro. However, RHAMM has neither any obvious signal peptide, nor a transmembrane domain, and the molecular basis for its localization remains undefined and a matter of controversy. Interestingly, previous data suggests an association of RHAMM and erk after PDGF-BB stimulation, and it has been suggested that RHAMM is a glycosyl-phosphotidyl inositol (GPI)-linked protein.

We therefore investigated whether RHAMM is a GPI anchored protein and its potential role in regulating injury and PDGF-BB mediated SMC migration. We show here that RHAMM is a critical requirement for PDGF-BB stimulated migration but not proliferation, and that it localizes to the cell surface via GPI modification. Most importantly, we show that RHAMM localizes to specific cholesterol-rich lipid domains in the plasma membrane of SMC, and its movement to the central sphingolipid core and interaction with caveolins via a specific caveolin-binding motif in RHAMM is a key requirement for propagating PDGF-BB signals for SMC migration and MAPK activation. These findings facilitate the development of screening assays to identify therapeutic agents which modulate a plethora of signaling events regulated by caveolins within lipid domains and facilitate elucidation of the involvement of RHAMM in cell migration, transformation and oncogenesis.

The following materials and methods are provided to facilitate the practice of Example II.

Reagents and Antibodies

PDGP-BB was obtained from R & D Systems (Minneapolis, Minn.). The rabbit polyclonal anti-RHAMM antibody (R36) was raised against a peptide (amino acids 586 to 605) encoded in the full-length murine cDNA for RHAMM and the IgG fraction was isolated. The specificity of this antibody for RHAMM and its ability to block HA binding to the receptor have previously been described. Caveolin antibodies were from BD Biosciences (Lexington, Ky.). Anti-urokinase receptor antibody was from American Diagnostica (Greenwich, Conn.). Phospho-p44/42 Map Kinase and p44/42 Map Kinase antibodies were from Cell Signalling Technology (Beverly, Mass.). Anti-PDGF Receptor Type B antibody was from Upstate Biotechnology (Lake Placid, N.Y.). AlexaFluor 488/594 antibodies were from Molecular Probes (Eugene, Oreg.). Percoll and OptiPrep for caveolar isolation were from Sigma. Mannosamine and Phosphatidylinositol-specfic phospholipase C. (PI-PLC) from Bacillus thuringiensis were from ICN (Costa Mesa, Calif.). EZ-link Sulfo-NHS-SS-Biotin and Streptavidin-agarose were from Pierce (Rockford, Ill.). Protein A and G agarose were from Invitrogen (Carlsbad, Calif.). Inositol-free media was from US Biological (Swampscott, Mass.), and ³H myo-inositol was from NEN (Boston, Mass.). Methyl-beta cyclodextrin was from Sigma Chemical Company (St. Louis, Mo.).

### Cell Culture

Primary cultures of rat aortic SMC were established as previously described. Briefly, thoracic aortae obtained from appropriately anesthetized animals were plated as 1 mm 3 explants in DMEM medium (Dulbecco's Modified Eagle Medium with 2 mM L-glutamine, 20 mM Hepes buffer, 1% antibiotic-antimycotic solution (penicillin G sodium 100 μg/ml, streptomycin sulfate 100 μg/ml, amphotericin B 0.25 μg/ml as Fungizone) with 20% fetal calf serum. SMC migrating out of these explants over 1-2 weeks were passaged once into DMEM medium with 10% FCS. Confluent cultures were slowly frozen to −80° C. in freeze mixture (95% FCS and 5% DMSO) and stored in liquid nitrogen. SMC used for experiments were thawed, maintained in a humidified incubator at 37° C. and 5% C0₂, and were used between passage numbers 2-4.

PDGF-BB and HA Stimulation

The medium of confluent SMC cultures was then changed to DMEM defined medium for 48 hours for quiescence. Migration of SMC at the leading edge of single scratch wounds of confluent cultures was determined using timelapse cinemicrography as previously described and summarized below. To observe the effect of HA on this migratory response to wounding, cultures were stimulated with varying concentrations of HA (0.1-100 μg/ml) and studied for 24 hours. For PDGF-BB stimulated HA synthesis experiments, quiescent SMC were stimulated with varying doses of PDGF-BB (2-100 ng/ml) for 24 hours. Media HA concentration was determined as described below. All experiments described below included 10 replicates and each experiment was repeated at least 3 times.

Antibody Blocking Studies

To determine the ability of R36 to block PDGF-BB-stimulated SMC migration, SMC cultures, after single scratch wounding, were exposed to exogenous PDGF-BB (10 ng/ml) with and without the presence of either R36 or rabbit IgG as a control (both at 100 μg/μl). The motility of SMC at the leading edge of wounds was followed using timelapse cinemicrography as described below.

Determination of Cell Migration

SMC plated in DMEM complete medium were allowed to grow to confluence. After overnight quiescence, a single scratch wound was made in the center of the culture plate using a sterile cell scraper and fresh defined medium was added The motility of cells at the leading edge of these wounds was determined using timelapse cinemicrography (Metamorph, Universal Imaging Corporation, West Chester, Pa.) as previously described. Briefly, cells were monitored using a Nikon TE-300 inverted microscope (Nikon Instruments, Inc., Melville, N.Y.) to which a video camera (Hamamatsu CCD, Inc., Hamamatsu City, Japan) was attached. At least 20 cells per high power field were imaged every hour and the mean distance traveled per frame was measured to obtain mean cell velocities.

Determination of HA Concentration

A modified ELISA assay was utilized for determination of HA concentration.

Immunofluorescence and Confocal Microscopy

Cells were grown to 60% confluency in 6-well dishes containing treated covers lips (Fisher). After 48 hour quiescence, cells were treated with 10 ng/ml PDGF-BB in fresh defined DMEM or fresh defined DMEM alone for 6 hours. The cells were fixed and permeabilized with methanol at −20° C. for 24 minutes and then washed with PBS. Cells were blocked with goat serum for 1 hour at room temperature, and then incubated with primary antibodies (caveolin-I M IgG 0.5 μg/ml; R36 5.0 μg/ml (for A10)) for 1 hour at room temperature. The PBS wash was repeated and followed by 30 minute incubation with secondary antibodies (Molecular Probes) at room temperature in the dark. Cells were washed with PBS, then distilled water, and finally mounted on slides with Fluormount. Slides were examined using Nikon and the Metamorph Imaging system).

### Electron Microscopy

Cells were grown to 60% confluence in 60 mm dishes and made quiescent in defined DMEM for 48 hours. PDGF stimulation was for 6 hours after which control and stimulated cells were fixed in 2% osmium tetroxide and embedded in epoxy resin.

### Electrophoresis and Immunoblot Analysis

Proteins were electrophoresed in either MES-SDS running buffer for SP-B or in MOPS-SDS buffer for SP-A and SP-D under reducing conditions at 200 volts for 35-45 min per the manufacturer's instructions (Invitrogen). Proteins were then transferred from the gel to a nitrocellulose membrane using the XCell II Mini-Cell and sandwich blot module (Invitrogen) in Bicine-10% methanol-0.01% SDS transfer buffer (Invitrogen) at 30 volts for 1 h. Blots were blocked for 1 h at room temperature with 5% nonfat milk and then incubated with primary antibody overnight at 4° C. Blots were then incubated with 1:5,000 goat anti-rabbit IgG-horseradish peroxidase for 1 h at room temperature. Signal was detected using the enhanced chemiluminescence kit (Amersham, Arlington Heights, Ill.), and blots were exposed to Kodak Biomax MS film. Blots were developed for varying lengths of time, and relative changes in protein content were determined by first scanning the blots using an Agfa Arcus II scanner and FotoLook SA scanning software into a Macintosh G4 Power PC computer. Semi-quantitative densitometric analysis of bands was accomplished using MacBAS version 4.2 (Fujifilm) after subtraction of background density.

Subcellular Fractionation

Cells were grown to 60% confluency in 150 mm dishes in DMEM complete medium and then quiesced for 48 hours. For PDGF-BB studies, media was then replaced with either fresh defined DMEM or 10 ng/ml PDGF in defined DMEM for 1, 6, 24, and 48 hours. For injury studies, several parallel scratch wounds were made using a sterile cell scraper and fresh defined DMEM was added for 1, 6, 24, and 48 hours. In both instances, cells were harvested identically. After washing 3 times with PBS on ice, cells were lysed in a solution containing 500 mM Na₂C0₃ (pH 11.0) with protease inhibitors for 10 minutes on ice. The cells were scraped from the plates, sonicated, and then centrifuged at 1000×g for 10 minutes. The supernatant was collected, sonicated, and equilibrated with an equal volume of 85% sucrose/MES buffer (MES buffer=25 mM A-morpholine-ethanesulfonic acid (pH 6.5) and 0.15 M NaCl for 2 hours at 40° C. This was then overlayed with 6 ml of 30% sucrose/MES buffer, then with 3.5 ml of 5% sucrose/MES 25 buffer, and finally ultracentrifuged at 34,000 rpm for 16-18 hr at 4° C. using a Beckman S W 40Ti rotor. The gradient was then separated into 1 ml fractionations taken from the top. Samples were ethanol precipitated and electrophoresed for western blotting as per NuPAGE protocol (Invitrogen).

Caveolar Fractionation

Caveolar fractionation was performed as previously described. Cells were washed in 0.25M sucrose, ImM EDTA, and 20 mM Tricine, pH7.8 (buffer A). Cells were collected by centrifugation at 1400 g for 5 min, then suspended in 1 ml of buffer A and homogenized. Homogenate was centrifuged at 1000 g for 10 min to collect the postnuclei supernatant (PNS). The PNS was then layered on 23 ml of 30% Percoll and centrifuged at 84000 g for 30 min in a Beckman Ti60 rotor. The plasma membrane fraction (a visible band 5.7 cm from the bottom of the centrifuge bottle) was collected and adjusted to a final volume of 2 ml with buffer A. This was then sonicated for 6s two successive times and mixed with OptiPrep (final concentration of 23% OptiPrep). The mixture was placed at the bottom of a Beckman SW 40 tube, overlayed with a 10-20% linear OptiPrep gradient, and then centrifuged at 52000 g for 90 min. The top 5 ml were transferred to a new SW40 tube and mixed with 4 ml of a 50% OptiPrep solution, containing 83% OptiPrep, 42 mM sucrose, 1 mM EDTA, and 20 mM Tricine, pH 7.8. This was overlayed with 1 ml of 15% OptiPrep solution and 0.5 ml of 5% OptiPrep solution prepared by diluting 50% OptiPrep solution with buffer A. The mixture was centrifuged at 52000 g for 90 min and caveolae/lipid raft membranes were collected from the band at the 5% interface.

Membrane Preparation

Cells grown to 60% confluency were quiesced for 48 hours and media was then replaced with either fresh defined DMEM or 10 ng/ml PDGF in defined DMEM for 6 hours. Plates were washed with calcium and magnesium free PBS and then incubated with 1 mM EDTA/PBS for 15 minutes at 37° C./5% CO₂. Cells were then scraped, collected, and centrifuged at 1000 RPM for 10 minutes. The pellet was resuspended in cold PBS and centrifugation repeated. The resulting pellet was sonicated in membrane preparation buffer: 0.25M sucrose/10 mM Hepes pH 7.1/0.5 mM DTT/10× protease inhibitor (Sigma). After sonication, samples were centrifuged at 1000 RPM for 10 minutes. The supernatant was ultracentrifuged at 200,000 g for 45 minutes. The resulting pellet was resuspended in 100 μl of membrane preparation buffer.

Immunoprecipitation

For standard experiment, 100 μg of lysate was used. Samples were pre-cleared with 50 μl of Protein G-agarose for 60 minutes at room temperature while rotating then centrifuged at 13000 g for 5 minutes. The supernatant was transferred to a new tube and incubated with 1 μg of antibody overnight at 4° C. while rotating. The samples were then incubated with 20 μl of Protein G-agarose for 1 hour at 4° C. while rotating. Beads were collected by centrifugation (1 minute at high speed) and then washed three times with lysis buffer (50 mM Tris, pH 8.0/140 mM NaCl/% Triton X-100/0.4% Deoxycholate/protease inhibitor cocktail (Boehringer Manheim). The beads were resuspended in at least 40 μl of 2× Novex Sample Buffer and western blot was performed as per NUPAGE protocol (Invitrogen). Primary and secondary antibodies were diluted (R36: 5 μg/ml, Caveolin-1: 0.25 μg/ml) and pre-incubated together over-night before use. For biotin labeling experiments, 6 μl of streptavidin-agarose was added to 25 μg of protein and incubated for 3 hours at 4° C. Beads were collected by centrifugation at 13000 g for 1 minute, washed and resuspended in Novex sample buffer as above.

### GPI-anchored Receptor Identification

Differential solubilization and temperature-induced Triton X-114 phase separation was performed as briefly described with 75-100 μg of membrane protein. Cells were grown to confluency and quiesced for 48 hours at which point cells were harvested for membrane protein. Some samples were incubated with PI-PLC for 1 hour at 37° C. before proceeding with Triton X-114 phase separation. PI-PLC was used at 0.375 U/100 μg membrane protein. In separate experiments Mannosamine-HCl was used at 10 mM during 6 hour PDGF-BB treatment.

For radiolabel experiments, cells were grown to confluency and quiesced for 48 hours in inositol-free media and then incubated with 4 μCi/mL of ³H myo-inositol during 6 hour PDGF-BB treatment. For surface biotinylation experiments, media was removed after treatment and replaced with 1 mg/ml biotin in defined media and incubated for 20 minutes at 37° C.

Caveolar Disruption

Caveolar disruption was performed as described below. Cells were grown to confluency in 150 mm dishes and quiesced for 48 hr. After 6 hr PDGF treatment, media was removed and replaced with 20 mm methyl-beta-cyclodextrin in TKM buffer (50 mM Tris-HCl, pH7.4, 25 mM KCl, 5 mM MgCl, 1 mM EDTA). Cells were incubated for 20-30 minutes at 37° C., and the cell-free supernatant was collected after centrifugation at 2000 g for 10 minutes.

### Results

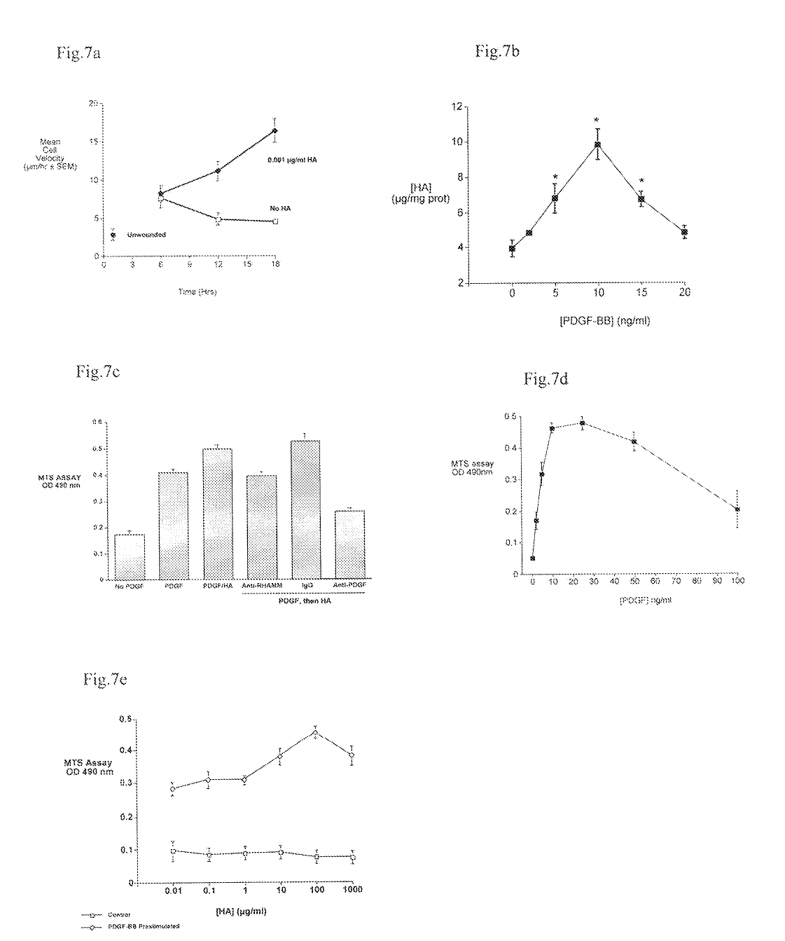

The effect of exogenous HA on SMC migration was tested after single scratch wounding. We previously demonstrated that the addition of 0.0 μg/ml of HA to sub-confluent SMC cultures increases their random migration and this effect is blocked by anti-RHAMM antibody (R36). In addition, SMC migration after single-scratch wounding was also blocked by this antibody. In the current study, addition of 0.001 μg/ml of HA to wounded cultures resulted in further stimulation of SMC that continued up to 18 hours (FIG. 7a, *p<0.0 1 vs. non-stimulated cells). HA was therefore able to stimulate the migration of SMC above and for longer than that induced by wounding itself.

In order to determine the optimal concentration of PDGF-BB required to simulate HA synthesis in SMC, the HA content of medium from quiescent SMC treated for 24 hours with various concentrations of PDGF-BB was determined using an ELISA like assay for HA. Resulting values were normalized to the total protein content of the cultures. FIG. 7b shows that a dose dependent response to PDGF-BB treatment, with maximal HA synthesis occurring at a dose of 10 ng/ml (*p<0.01 vs. control). These data are in agreement with previous studies of PDGF-BB regulation of HA synthesis (Heldin P et al., Biochem J. Mar. 15, 1998; 258(3):919-22). We therefore used this concentration of PDGF-BB for all subsequent experiments.

Since PDGF-BB (10 ng/ml) stimulated maximal HA accumulation in the medium of SMC cultures, and exogenously added HA increased SMC migration, we determined the effect of PDGF-BB treatment and role of RHAMM in SMC migration after wounding. PDGDF-BB increased the mean cell velocity of SMC at the leading edge of wounds by two-fold compared to the non-stimulated control (FIG. 7c, *p<0.01 vs. no PDGF-BB). Treatment of these cultures with anti-RHAMM antibody (R36), but not normal IgG, completely blocked increased cell migration induced by PDGF-BB (FIG. 7c, **p<0.01 vs. normal IgG) suggesting that RHAMM is required for and can fully account for PDGF-BB-stimulated SMC migration after wounding.

In order to determine the effect of PDGF-BB on SMC proliferation, 80% confluent and quiescent cultures of SMC were stimulated for 72 hours with varying doses of PDGF-BB (2-50 ng/ml) and cell number was determined using the MTS assay as described in Methods. A bell-shaped dose response curve was obtained with maximum stimulation of SMC proliferation at 10-25 ng/ml (FIG. 7d, *p<0.01 vs. control).

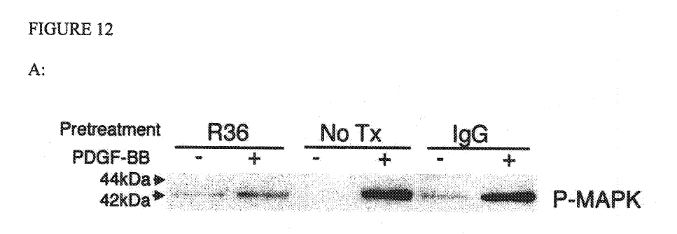

The effect of HA on SMC proliferation and modulation by PDGF-BB was also examined. SMC that were either maintained in serum-free medium or pre-stimulated with 10 ng/ml PDGF-BB for 24 hours were exposed to HA in serum-free medium at concentrations of 0.1-1000 μg/ml for 48 hours. SMC showed no proliferative response to any concentration of HA in the absence of pre-stimulation with PDGF-BB (Control, FIG. 12). As expected, baseline proliferation was increased in SMC cultures following exposure to 10 ng/ml PDGF-BB for 24 hours (FIG. 7e, *p<0.01 vs. non-stimulated cells). After pre-stimulation with PDGF-BB, SMC cultures demonstrated a further 25% increase in proliferation in response to HA with maximal response at 100 μg/ml (FIG. 7e, **p<0.05 vs. PDGF-BB-prestimulated but not HA-treated cells). In order to determine the contribution of RHAMM to SMC proliferation induced by HA, SMC cultures were treated with R36 throughout the pre-stimulation period with PDGF-BB and subsequently during HA stimulation. Non-immune IgG was used as a negative control and a blocking anti-PDGF-BB antibody was used as a positive inhibitory control. Cell number was determined by MTS assay 48 hours after HA stimulation with and without antibody treatments. As noted before, PDGF-BB pretreatment increased baseline proliferation, FIG. 7e*p<0.01 vs. no PDGF), and HA stimulated a further 25% increase in proliferation, FIG. 7e**p<0.05 vs. PDGF, no HA). Anti-RHAMM antibody completely blocked the HA-mediated increase in SMC proliferation, but did not have any effect on PDGF-BB-stimulated increase in baseline proliferation of non-HA-stimulated cultures, FIG. 7e***P<0.05 anti-RHAMM vs. normal IgG). Anti-PDGF-BB antibody blocked 50% of the PDGF-mediated increase in baseline proliferation, FIG. 7e#p<0.01 anti-PDGF vs. normal IgG).

RHAMM Associates with Lipid Domains and Caveolin upon PDGF-BB Stimulation

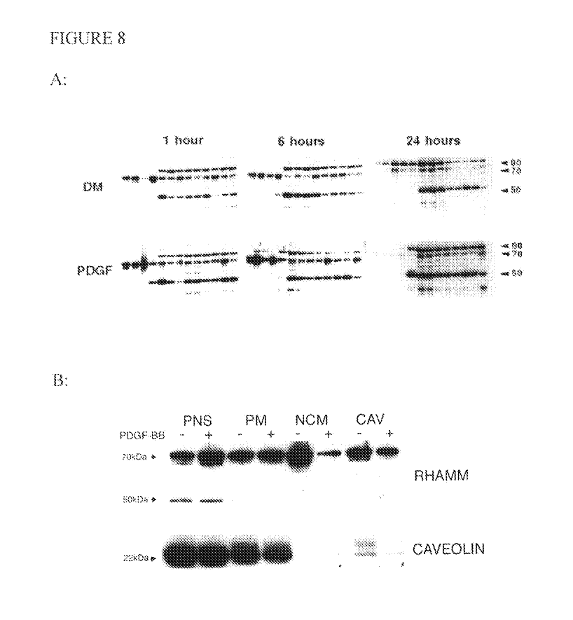

Given that the components of PDGF-BB mediated signaling are found in caveolae, that HA has been localized to caveolae in SMC, and that RHAMM was required for PDGF-BB stimulated SMC migration after wounding, we examined the localization and molecular interactions of RHAMM at the cell surface. Non-detergent sucrose gradient fractionation was performed on a time course study of quiescent vs. PDGF-BB treated SMC cultures (FIG. 8a). Immunoblot analysis revealed three major bands for RHAMM, approximately 50 kda, 70 kda, and 95 kda, all of which demonstrated increased expression with PDGF-BB treatment. However, only the 70 kda band was present in low density, buoyant fractions by 1 hour. While expression of the 70 kda band decreased over time in untreated cultures, its expression increased to a maximum at 6 hours and declined up to 24 hours. We also noted transient expression of the 95 kDa band in low density fractions after 6 hours of PDGF-BB treatment and this too declined over 24 hours. Expression of the 50 kDa remained constant throughout the time course.

To further define the compartment of the plasma membrane in which RHAMM resides in the presence and absence of PDGF-BB treatment, sequential fractionation was undertaken to separate caveolar and non-caveolar membranes. The distribution of RHAMM and caveolin was then determined by immunoblotting (FIG. 8b). As expected. caveolin was present in the postnuclear supernatant and plasma membrane, was absent from the non-caveolar fractions, and was enriched in the caveolar fraction.

Immunoblotting for RHAMM in the same samples showed 50 kDa, 70 kDa, and 95 kDa bands in the post-nuclear supernatant. Interestingly, only the 70 kDa band was present in the plasma membrane, non-caveolar membrane, and caveolar membrane fractions. While samples both with and without PDGF-BB stimulation were studied, the procedures for membrane purification are not quantitative and relative distribution in the presence or absence of growth factor cannot be interpreted.

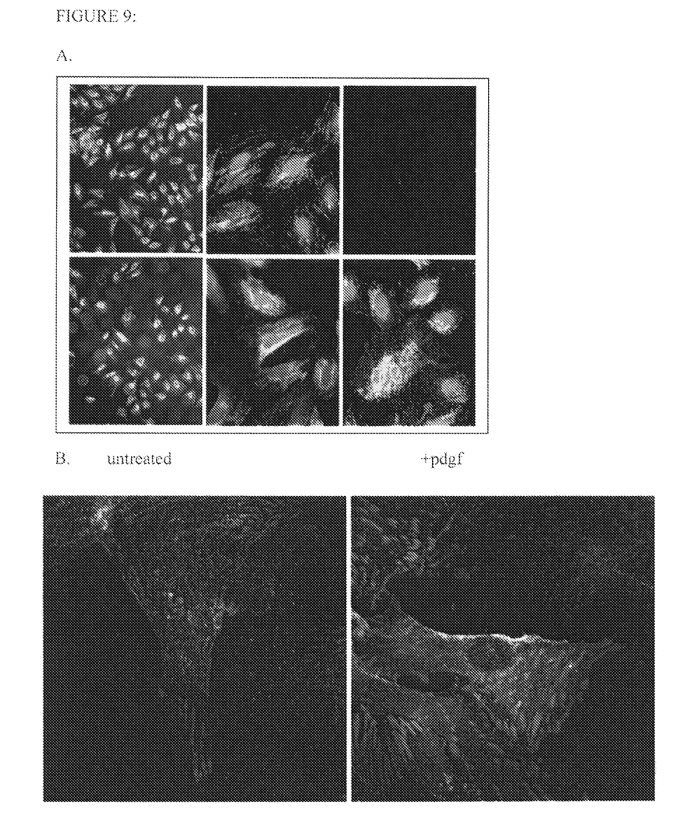

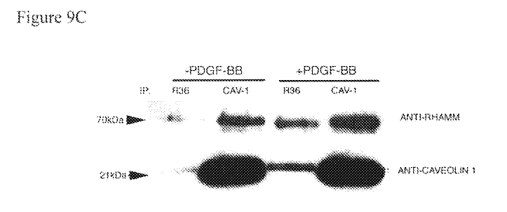

Next, we utilized multi-channel confocal immunofluoresence microscopy to visualize RHAMM and caveolin-1 localization in SMC cultures in the presence or absence of PDGF-BB treatment (FIG. 9). Quiescent, untreated SMC expressed caveolin-1 (red) on their surface, and RHAMM (green) expression was primarily intracellular (FIG. 9a). With 6 hour PDGF-BB treatment, both RHAMM and caveolin-1 expression was increased intracellularly as well as on the cell surface, and extensive co-localization (yellow) was observed at the cell membrane (FIG. 9b). This co-localization did not occur at focal adhesions since no overlay was observed with vinculin (purple) even though focal adhesions were increased with PDGF-BB treatment (FIGS. 9a & b). To confirm these findings, double-label immunogold electron microscopy showed distinct co-labeling of RHAMM and caveolin-1 on the surface and in caveolar invaginations.

To test the association of RHAMM:caveolin, cell lysates were immunoprecipitated for RHAMM and caveolin-1 after six hours of PDGF-BB treatment. Immunoblot analysis for RHAMM showed that the 70 kda form of RHAMM co-precipitated with caveolin-1 antibody and that this association increased with PDGF-BB treatment (FIG. 9c). Immunoblotting with anti-caveolin antibody resulted in a characteristic 21 kDa band in all precipitates that increased with PDGF-BB treatment. Similar results were achieved with caveolin-2 and -3 precipitation (data not shown). Conversely, IP with R36 co-precipitated caveolin-1 and this association inceased with PDGF-BB treatment.

It has been suggested that co-immunoprecipitation may only represent physical proximity of proteins within the same domain or complex. Therefore, peptides were used to interfere with RHAMM:caveolin to establish direct protein-protein interactions. Caveolin binding proteins contain a consensus caveolin-binding motif (ΦxΦxxxxΦxxΦ), which interacts with the scaffolding domain (aa 82-101) found in caveolin. Upon, scanning the RHAMM protein sequence, we discovered a putative caveolin-binding motif (KWRLL YEEL YDKTKPFQ). Peptides were created against this RHAMM:caveolin binding motif (RM) and the caveolin scaffolding domain (CAV) and then fused to an antenapedia sequence (RQIKIWFQNRRMKWKK) to facilitate internalization as described previously by Sessaet al. SMC cultures were treated with peptides or corresponding scrambled peptides (RMX and CAVX) for 24 hours prior to and during followed by 6 hour PDGF-BB treatment. Whole cell lysate was collected and immunoprecipitated for RHAMM. Immunoblot for caveolin-1 showed that decreased co-immunoprecipitation of RHAM:M and caveolin-1 occured with either RHAMM or caveolin peptide treatment.

RHAMM Associates with a Glycosyl-Phoslphatidyl-Inositol Anchored Protein such as UPAR

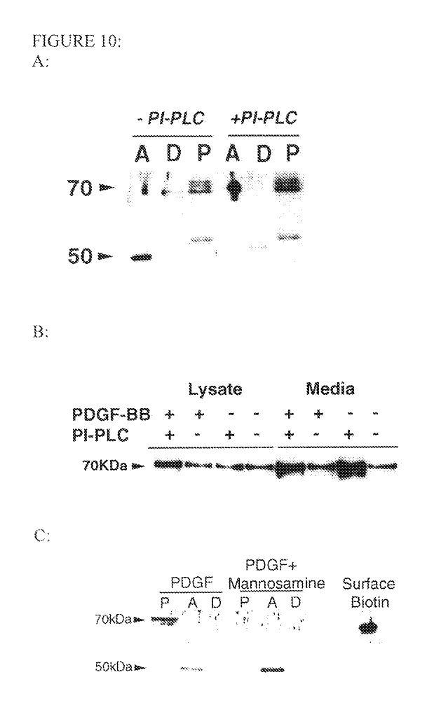

To date, the mechanism by which RHAMM reaches the cell surface has been unclear. RHAMM does not have a transmembrane domain or obvious signal sequences, and although no previous data are published, it has been suggested that RHAMM is GPI-anchored. To explore this possibility, we began with Triton X-114 fractionation as described in Methods. This non-ionic detergent can be used to distinguish between, soluble/hydrophilic proteins, integral/transmembrane proteins, and GPI-anchored proteins in cell lysates. Immunoblot analysis of untreated SMC lysates showed that 70 kda RHAMM separated to the pellet phase, suggesting that it is GPI-anchored (FIG. 10a). To further confinn GPI modification, SMC cultures were first treated with phosphotidyl inositol-specific-phospholipase C (pI-PLC), an enzyme that specifically cleaves GPI anchors from cell surface proteins. As expected, with PI-PLC treatment, the majority of 70 kDa RHAMM shifted from the pellet fraction to the aqueous fraction. Surface localization and confirmation of PI-PLC sensitivity was accomplished by cell surface biotin labeling prior to PI-PLC treatment. After six hour PDGF-BB treatment, cells were surface-biotin labeled, then treated with PI-PLC for one hour. Cell lysates and corresponding cultured media were collected and immunoprecipitated with streptavidin-agarose. Immunoblot analysis showed that 70 kDa RHAMM was indeed cleaved and released into the media with PI-PLC treatment (FIG. 10b, lane 3 vs 7 and lane 1 vs 5). Interestingly, this release occurred even without PDGF-BB or PI-PLC treatment, suggesting that RHAMM is constitutively shed from the cell surface (FIG. 10b, lane 4 vs lane 8).

GPI-anchors are invariably followed by a tri-mannose core, the incorporation of which can be competitively disrupted by the amino sugar mannosamine. Therefore, GPI modification of RHAMM was further tested with mannosamine treatment. As expected, co-treatment of cells with PDGF-BB and mannosamine for six hours completely inhibited the presence of RHAMM in the pellet phase after Triton X-114 triple fractionation (FIG. 10c). In all experiments, the 50 and 95 kDa forms of RHAMM were not biotin labeled, were not PI-PLC sensitive, or shed into the media.

Movement of RHAMM Within Lipid Raft Subdomains

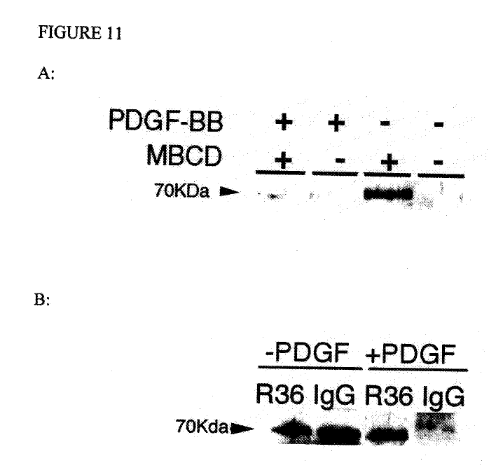

Since the GPI anchor links to a hydrophobic lipid tail that inserts into the exoplasmic leaflet of the cell membrane, a liquid ordered arrangement is necessary to maintain its integrity. Hence, lipid microdomains and caveolae are often enriched with GPI-anchored proteins. Methyl-beta-cyclodextrin (MBCD), a cell surface-specific cholesterol-extracting agent, can be used to disrupt lipid rafts and caveolae to release endogenous GPI-anchored proteins (Ilangumaran, Madore, McCabe). Recent studies have suggested that lipid domains within the plasma membrane contain distinct subdomains consisting of a central phospholipid-rich, highly-ordered sphingolipid core surrounded by a cholesterol-rich, semi-ordered annulus. Proteins in the cholesterol-rich annulus are sensitive whereas those in the sphingolipid core are resistant to release by MBCD. SMC cultures were treated with MBCD for 30 minutes, and the cell free supernatant was collected. The supernatant was run by volume on SDS-PAGE and immunoblotted for RHAMM and urokinase-plasmingen activator receptor (uPAR), a defined GPI-anchored protein previously shown to be present in lipid domains. Without MBCD, RHAMM was not released into the supernatant in detectable amounts, irrespective of PDGF-BB treatment (FIG. 11a, lane 2 vs 4). With MBCD treatment in the absence of PDGF-BB treatment (FIG. 11a, lane 3) 70 kDa RHAMM was released into supernatant. However, MBCD treatment failed to release RHAMM when cells were treated with PDGF-BB (FIG. 11a, lane 3), suggesting that, upon growth factor stimulation, RHAMM becomes more tightly associated with the membrane by shifting to the MBCD-resistant sphingolipid-rich region of the lipid domain. MBCD-mediated UPAR release remained unaffected regardless of treatment.

Since R36 blocked PDGF-BB-stimulated SMC migration, we explored the effect of this blocking antibody on the ability to release RHAMM using MBCD. FIG. 11b shows the release of RHAMM with MBCD with and without PDGF-BB treatment. In the absence of PDGF-BB, RHAMM was released into the supernatant regardless of R36 or IgG treatment. As expected, with PDGF-BB stimulation, RHAMM became MBCD-resistant in IgG treated cultures. In contrast, R36 treatment of cultures was associated with continued MBCD-mediated release of RHAMM even in the face of PDGF-BB treatment, suggesting that R36 prevented the movement of RHAMM into the central sphingolipid core.

Previous co-immunopreciptitation studies have shown that RHAMM associates with, and contains recognition sequences for, erk and src as well as additional serine/threonine kinases. Furthermore, RHAMM is required for PDGF-mediated erk activation and tyrosine phosphorylation of erk 1,2. We therefore explored the effect of antibody and peptide treatment on erk phosphorylation. PDGF-BB stimulated SMC cultures were treated for 10 minutes with either R36 or normal IgG. Whole cell lysate was collected and immunoblotted for total and phosphorylated-erk (FIG. 12a). PDGF-BB treatment resulted in increased erk phosphorylation both in untreated and IgG-treated cultures, but not in R36-treated cultures suggesting that RHAMM is involved in PDGF-BB mediated erk phosphorylation. In separate experiments, SMC cultures were pretreated with peptides (RM and CAV) or corresponding scrambled peptides (RMX and CAVX) for 24 hours followed by 10-minute PDGF-BB treatment. Whole cell lysate was collected and immunoblotted for total and phosphorylated-erk. Non-peptide treated cells and cells treated with scrambled peptides that were exposed to PDGF-BB increased erk phosphorylation over that of untreated control cells. However, with either RM or CAV peptide pretreatment, erk phosphorylation was inhibited.

### Discussion

Regulation of embryonic development and responses to tissue injury are under the intricate control of growth factors. Growth factor signaling appears to be mediated via specialized lipid-rich domains within the plasma membrane where signal transduction molecules are concentrated. Caveolae, cholesterol and sphingolipid-rich invaginations of the plasma membrane, are created by the presence of caveolins, a family of three proteins that keep signaling molecules in the quiescent or “off” state. A number of growth factor receptors have been shown to interact with caveolins, including transforming growth factor (TGF)-B Receptor I and platelet-derived growth factor (PDGF)-B receptor. In addition to ligand:receptor interactions of growth factors, hyaluronan (HA) and its receptors are emerging as critical regulators of growth factor activation and signaling. RHAMM was first isolated as a 56-58 kDa protein from the supernatant media of non-confluent embryonic chick heart fibroblasts and shown to regulate their ruffling and migration. A number of critical functions, including the motility of thymocytes, lymphocytes, hematopoetic progenitor cells, malignant B lymphocytes, fibroblasts, smooth muscle cells, endothelial cells and macrophages, as well as regulation of MAP kinase-mediated proliferative responses and cellular transformation have been ascribed to it. Study of this intriguing molecule has been the subject of controversy with two diametrically opposing views on whether RHAMM is expressed on the cell surface or not. Cell surface localization of this receptor has been demonstrated by subcellular localization, surface labeling, antibody blocking and F ACS analysis. However, studies in adherent cells transfected with full length RHAMM cDNA showed an exclusively intracellular protein that interacts with the cytoskeleton, and other studies of RHAMM expression have failed to demonstrate surface expression.

The structure of RHAMM, as predicted by its full-length cDNA, does not reveal either an obvious signal peptide or a transmembrane domain. Therefore, we investigated the hypothesis that RHAMM is a glycosyl-phosphatidyl inositol (GPI) anchored protein. A GPI anchor consists of a tri-mannose core, followed by an ethanolamine that is covalently linked to the C-terminal amino acid of the attached protein. Disrupting this core using synthase mutants, sugar analogs, or mannosamine blocks the formation of the GPI anchor and thereby prevents proteins from reaching the cell surface. The tri-mannose core is connected to the glycerol backbone of the membrane associated lipid moiety via a phosphate bridge, which is susceptible to cleavage by phosphatidylinonsitol-specific phospholipase C (PI-PLC). Protein release following PI-PLC treatment is another criterion for the presence of a GPI anchor. Our findings of a block in GPI modification in the presence of mannmosamine, sensitivity to PI-PLC, and Triton X-114 differential solubilzation into the GPI-rich pellet phase strongly support that the 70 kDa form of RHAMM is a (GPI)-modified protein. Direct ethanolamine labeling of RHAMM confirms that RHAMM is GPI modified and that these associations are not the result of the interaction of RHAMM with another GPI-anchored protein. Further, surface biotinylation and PI-PLC release into the medium confirms that GPI-modified 70 kDa RHAMM is expressed at the cell surface.

While GPI-anchored proteins have previously been shown to interact with caveolin, and our studies confirm RHAMM:caveolin interaction, how this occurs is still unknown since GPI-anchored proteins are specifically found in the exoplasmic leaflet of the cell membrane. It has been suggested that the fatty acid residues present in the GPI tail are long enough to penetrate the bilayer and directly interact with cytoplasmic signaling molecules. Also, regulated GPI hydrolysis in response to hormones or growth factors can lead to signal transduction. Additionally, several GPI-anchored proteins have been shown to act as adaptor proteins. For example, caveolin associated −1 integrin signaling occurs through interaction with the GPI-anchored urokinase type plasminogen activator receptor uPAR. As integrins activate by ligand-mediated clustering, uPAR moves into the complexes with caveolin associated signaling molecules. Removal of uPAR impairs the association. By using function blocking monoclonal antibodies, Pilarski's group has shown that RHAMM contribution to the mobilization of hematopoietic progenitor cells is regulated by β1 integrin interaction with the ECM since both RHAMM and β1 integrin antibodies were able to block motility, but only −1 integrin antibody was able to inhibit adherence of cells on fibronectin. However, our finding that UPAR is not differentially extracted by MBCD suggests that RHAMM:caveolin interactions are different from those involving UPAR. It is possible that UPAR:caveolin:β1 integrin regulate ECM interactions whereas RHAMM regulates the propagation of growth factor signals in the motile phenotype.

In the present study, we demonstrate a critical requirement for RHAMM in PDGF-BB stimulated SMC migration after single scratch wounding. Both single scratch wounding and PDGF-BB stimulation of SMC cultures resulted in increased surface localization of RHAMM which, based on antibody blocking studies, is a critical requirement for HA mediated responses. PDGF-BB-mediated SMC migration is entirely dependent on RHAMM:HA interaction as it can be fully blocked with anti-RHAMM antibody. However, this antibody blocked HA- but not PDGF-BB-stimulated proliferation suggesting the presence of additional autocrine or post-receptor signaling mechanisms regulating SMC proliferation in response to PDGF-BB. We have previously demonstrated differential functions of RHAMM and CD44 in endothelial cells where RHAMM-regulated migration and CD44-regulated proliferation and adhesion to HA. HA however has previously been implicated in SMC proliferation. HA is upregulated in the atherosclerotic lesions of atherosclerosis-prone apoe deficient mice, and proliferation of cultured primary aortic SMC from these mice is stimulated by low molecular weight HA whereas high molecular HA inhibits SMC proliferation (Pure). Further, Jain et al. reported that HA stimulates rat vascular SMC proliferation in a dose dependent manner. The doses of HA used to stimulate proliferation in both their studies and this report are similar (100 μg/ml). In the studies by Jain et al., proliferation in response to HA was observed without growth factor pre-stimulation. However, in our studies, no proliferative response was observed in the absence of pre-stimulation with PDGF-BB. We note that the SMCs in their studies were maintained in 0.4% FCS for quiescence whereas serum-free medium was used in ours. It is possible that 0.4% FCS is sufficient to pre-stimulate these cells to respond to HA.

Our data support the association of 70 kDa RHAMM with caveolins via a specific sequence in RHAMM and the caveolin scaffolding domain. Specifically, peptides mimicking a putative caveolin-binding site in RHAMM (KWRLL YEEL YDKTKPFQ) and the caveolin scaffolding domain decreased co-immunoprecipitation of RHAMM with caveolin. It is currently thought that many signal-transduction cascades are initiated within caveolae. The plethora of molecules that bind to caveolin-1 include actin-cytoskeleton-related, G-protein- coupled receptors, growth-factor receptors, channels, and cytoplasmic signaling molecules such as src-like kinases, eNOS, and MAP kinase. All known proteins that interact with the caveolin scaffolding domain (aa 82-101) contain a consensus caveolin binding motif, ΦxΦxxxxΦxxΦ, where Φ is any aromatic amino acid. Functionally, caveolin negatively regulates signaling pathways by maintaining bound molecules in an inactive state. For example, both in vitro and in vivo, eaveolin-1 can inhibit signaling along the p42/44 MAPK cascade by acting as an endogenous inhibitor of EGFR, MEK and erk.

Signaling via RHAMM has recently been reviewed. Binding of HA to membrane RHAMM results in a transient burst of protein tyrosine phosphorylation and focal adhesion turnover, activation of src, and regulation of the erk kinase cascade through ras. Indeed, when cell cultures were pretreated with R36, MAPK phosphorylation was blocked after PDGF-BB treatment. This transduction pathway appears to be mediated by caveolin since pretreatment of cell culture with peptides mimicking the caveolin scaffolding domain or the caveolin binding motif in RHAMM also blocked MAPK phosphorylation after PDGF-BB treatment. Although RHAMM has been shown to co-immunoprecipitate with erk, it is unlikely that our results are due to blocking of this intracellular association since R36 is not internalized and our results show cell surface changes with caveolin.

The importance of spatial signaling and receptor movement within sub-domains has been described previously. Hoessli's group defines sub-domains within lipid microdomains by their detergent solubility and cyclodextrin sensitivity. The significance of these sub-domains is not well defined, but they may allow subtle regulation of cell surface signaling altering protein interactions in response to extracellular stimuli. For example, it is already known that in unstimulated cells, EGF receptors are concentrated in caveolae and the critical steps in the EGFR pathway take place there. The down regulation of EGFR signal coincides with the exit of EGFR from caveolae. A similar scheme has been described for the PDGF receptor, but exact details remain controversial. Our data suggest that RHAMM normally resides in the cholesterol annulus, and upon stimulation with PDGF-BB, shifts to the sphingolipid-rich core of the lipid microdomain. Once in the core, RHAMM becomes detergent insoluble and cyclodextrin resistant. Since anti-RHAMM antibody treatment results in a continued ability to release of RHAMM by MBCD, prevents RHAMM:caveolin association, and blocks downstream erk phosphorylation effects of growth factor stimulation, RHAMM movement to the sphingolipid core is required for caveolin association and propagation of the PDGF-BB signal for MAPK activation and motility.

Our studies add to a growing list of mechanisms by which HA receptors regulate growth factor activation and signaling at the cell surface. First, Stamenkovic's group demonstrated the role of HA:CD44 interaction in localizing matrix metalloproteinase-9 (MMP-9) to the cell surface of melanoma cells as a mechanism for activation of latent TGFβ1. Second, in a human breast cancer cell line, Bourguignon et al. demonstrated a direct interaction of the cytoplasmic tail of CD44v3 with TGFβ Receptor I. HA binding to CD44 resulted in phosphorylation of CD44, interaction with ankyrin, a component of the cytoskeleton, and downstream signaling through Smad3. The interactions of CD44, TGFβR1 and ankyrin resulted in enhanced cell migration. While CD44 has previously been localized to lipid domains and caveolae, it is unclear at present if eaveolae are involved in CD44-mediated processes.

The findings presented herein have far reaching implications for growth factor regulation of homeostatic and pathologic processes. Based on the unique results of the requirement of RHAMM:caveolin association for PDGF-BB mediated cell migration and signaling and the ability of R36 to block multiple chemoattractants in multiple cell types, we propose that RHAMM association with caveolin is required for appropriate release and interaction of signal transduction molecules. Previous studies showed that overexpression of RHAMM is transforming and required for ras transformation. These studies were controversial since a partial cDNA for RHAMM was used. We are currently evaluating isoforms of RHAMM in macrophages, and preliminary results demonstrate no differences between full length and 5′ truncated forms of RHAMM with respect to mediation of HA signals (unpublished results). Genetic deletion of RHAMM in mice predisposed to gastrointestinal and aggressive fibromatosis tumors attenuates the formation of aggressive fibromatosis, suggesting that RHAMM regulates proliferation of cells with sparse cell-cell contacts. Further, high levels of RHAMM expression in primary tumors of human breast cancer correlates with overexpression of ras and erk and poor outcome in disease progression.

The RHAMM transforming phenotype is remarkably similar to the phenotype of immortalized cultured cells, in which caveolin levels are down-regulated. Perturbed caveolar function in transformed cells results in loss of cell cycle control as evidenced by failure to recognize normal contact inhibition. Furthermore, it is known that the caveolin-1 gene localizes to a suspected tumor suppressor locus that is often deleted in human cancers. In fact, Schnitzer and Lisanti showed that replacing the caveolin-1 gene in human cancer cells causes them to revert to normal. Caveolin-1 knockout mice demonstrate cardiovascular defects, nitric oxide abnormaltities, and lung pathology consistent with hyperproliferation. Potentially, overexpression of RHAMM would result in excess binding of caveolin, release of signal transduction molecules, thereby mimicking the absence of caveolin. Since R36 has previously been shown to block multiple signals for cell migration in multiple cell types, it is likely that the findings we report here are generalizable to other growth factors and cell types. Indeed, we are engaged in studies in endothelial cells and macrophages to test these predictions. Elucidation of the mechanisms of HA receptor regulation of growth factor signaling may define their role during embryogenesis and develeopment as well as allow the development of novel therapeutic interventions for a wide variety of diseases caused by aberrant growth factor signaling, including neointimal formation, angiogenesis, wound healing and tumor establishment and progression.

While the invention has been described in detail and with reference to specific examples thereof, it will be apparent to one skilled in the art that various changes and modifications can be made therein without departing from the spirit and scope thereof.

1. A method for identifying agents which modulate RHAMM:Caveolin:Lipid raft meditated interactions comprising:
a) providing cells which express RHAMM;
b) placing cells under conditions known to alter RHAMM expression level or cellular localization;
c) incubating the cells of b) in the presence and absence of said agent; and
d) determining whether said agent alters RHAMM expression level or localization relative to cells which are not contacted with said agent, thereby identifying agents which modulate RHAMM:Caveolin:Lipid raft interactions.

2. The method of claim 1, wherein said agent inhibits said interactions.

3. The method of claim 1, wherein said agent increases said interactions.

4. The method of claim 1, wherein said conditions known to alter RHAMM expression level or cellular localization are selected from the group consisting of treatment with growth factors, treatment with HA, conditions resulting in tissue injury conditions which modulate angiogenesis, conditions which promote tumorigenesis and metastasis and conditions which invoke an inflammatory response.

5. The method of claim 1, wherein said cells are inflammatory cells and said agent increases or decreases surface expression levels of the 70 kDA form of RHAMM.

6. The method of claim 4, wherein said cells are in a mammal subjected to conditions resulting in tissue injury and said agent alters the number of inflammatory cells that migrate to said injury site.

7. The method of claim 4, wherein said cells are in a mammal subjected to conditions resulting in tissue injury and said agent alters inflammatory cell chemotaxis.

8. The method of claim 4, wherein said cells are in a mammal subjected to conditions resulting in tissue injury and said agent alters inflammatory cell motility.

9. The method of claim 1 wherein said agent disrupts RHAMM-GPI anchor interactions.

10. A method of inhibiting escalation of a tissue injury, said method comprising treating a mammal at an onset of the tissue injury with an agent which inhibits RHAMM:caveolin interaction.

11. A method of inhibiting escalation of a tissue injury, said method comprising treating a mammal at the onset of the tissue injury with an agent which inhibits interaction between RHAMM and hyaluronan (HA) oligosaccharides.

12. The method of claim 10, wherein the agent is a RHAMM antibody or a peptide mimicking a caveolin binding site in RHAMM.

13. The method of claim 11 wherein the agent is a peptide mimicking an HA binding site in RHAMM.

14. The method of claim 12, wherein the peptide mimicking a caveolin binding site in RHAMM is KWRLL YEEL YDKTKPFQ (SEQ ID NO: 5).

15. The method of claim 13, wherein said peptide mimicking an HA binding site in RHAMM is RGGGRGRRR (SEQ ID NO: 7).

16. The method of claim 1, wherein said cells are present in a mammal.


SEQUENCE LISTING (7 sequences)

SEQ ID NO: 1 - DNA, 21 bp, Artificial Sequence
  Primer
gttggttggttggaaaaatct

SEQ ID NO: 2 - DNA, 27 bp, Artificial Sequence
  Primer
gcagcagttcgggttgccttctttcaa

SEQ ID NO: 3 - DNA, 30 bp, Artificial Sequence
  Primer
gaaatagaagatcttaaactggagaatttg

SEQ ID NO: 4 - DNA, 30 bp, Artificial Sequence
  Primer
caaattctccagtttaagatcttctatttc

SEQ ID NO: 5 - protein, 17 aa, Artificial Sequence
  Synthetic Sequence
KWRLLYEELYDKTKPFQ

SEQ ID NO: 6 - protein, 16 aa, Artificial Sequence
  Synthetic Sequence
RQIKIWFQNRRMKWKK

SEQ ID NO: 7 - protein, 9 aa, Artificial Sequence
  Synthetic Sequence
RGGGRGRRR